(19) United States
(12) Patent Application Publication
(10) Pub. No.: US 2019/0307140 A1
(43) Pub. Date: Oct. 10, 2019

(54) STREPTOCOCCUS THERMOPHILUS FERMENTATION PROMOTING AGENT

(71) Applicant: MEIJI CO., LTD., Tokyo (JP)

(72) Inventors: Keisuke FURUICHI, Odawara-shi, Kanagawa (JP); Emi TOYAMA, Odawara-shi, Kanagawa (JP); Yoshimi MAKABE, Odawara-shi, Kanagawa (JP)

(21) Appl. No.: 16/309,519

(22) PCT Filed: Jun. 16, 2017
(86) PCT No.: PCT/JP2017/022287

§ 371 Date: Dec. 13, 2018

(30) Foreign Application Priority Data
Jun. 16, 2016 (JP) 2016-120216

(51) Int. Cl. A23C 9/123; A23L 2/38; C12N 1/20

(52) U.S. Cl. CPC A23C 9/1238; A23L 2/382; C12N 1/20; C12N 2500/12; A23V 2002/00; C12N 2500/34; A23Y 2240/75

(57) ABSTRACT

The present invention provides a lactic acid bacterial fermentation-promoting agent suitable for use in food production. The present invention relates to a fermentation promoting agent for Streptococcus thermophilus, comprising a solution prepared by exposing an alkaline solution comprising a reducing sugar to a temperature in the range of 5° C. or more and 135° C. or less to induce a color reaction of sugar, and a production method thereof; and a method for promoting fermentation for Streptococcus thermophilus using the fermentation promoting agent and a method for producing a fermented food such as fermented milk.

### TECHNICAL FIELD

The present invention relates to a fermentation promoting agent for Streptococcus thermophilus using a sugar-alkaline solution.

### BACKGROUND ART

Lactic acid bacteria have been used for the production of fermentation products including various foods. Promoted growth and fermentation of lactic acid bacteria provide significant industrial benefits in view of the streamlining of growth and fermentation process of lactic acid bacteria. Among lactic acid bacteria, Streptococcus thermophilus is used for the production of many fermented foods such as yogurt, and the promotion of Streptococcus thermophilus fermentation is particularly important. On the other hand, palatability is also an extremely important factor for foods including yogurt, and thus a fermentation promoting agent which has an adverse effect on the taste is not desirable. Given the use for food production, it is also important that the fermentation promoting agent can be produced inexpensively and that it exhibits a fermentation promoting effect even in a small amount. It would be useful to exhibit the effect when used in a small amount, because the existing production equipment can be used without reinforcement of equipment such as a facility for adding a fermentation promoting agent.

As technologies for promoting the growth and fermentation of lactic acid bacteria, there are a lactic acid bacterial growth-promoting agent containing, as an active ingredient, acidic buttermilk containing dead cells of lactic acid bacteria (Patent Literature 1); a lactic acid bacterial growth-promoting agent comprising agar having a reducing sugar content and a weight average molecular weight adjusted in certain ranges (Patent Literature 2); and a method for promoting the growth of gram-positive bacteria such as lactic acid bacteria using an extract derived from Musa species (Patent Literature 3). However, there is still a need for the development of a lactic acid bacterial fermentation-promoting agent that shows an effect even when used in a small amount, can be prepared inexpensively and has little influence on the taste.

Patent Literature 4 describes a method for producing lactic acid by culturing an alkalophilic lactic acid bacterial strain L-120 belonging to Enterococcus in a culture medium containing a saccharified cellulose solution (pH 9 to 11). However, Patent Literature 4 does not describe a method for promoting fermentation of Streptococcus thermophilus.

Meanwhile, sugars such as glucose are known to cause a color reaction under alkaline conditions or under heating conditions. This color reaction changes the color of a sugar-containing material to brown to black (i.e., to be stained). The color reaction of sugar is believed to be induced by any one or a combination of two or more of various reactions such as caramelization (mainly by heating at or near the melting point of each sugar of about 100° C. to 200° C. or at a temperature exceeding it), Maillard reaction (aminocarbonyl reaction; by reaction with amino compound); an alkali isomerization reaction (Lobry de Bruyn and Alberda van Ekenstein transformation reaction) and the like (see, e.g., Non Patent Literature 1), depending on the reaction conditions. It is believed that the color reaction of sugar produce many types of substances, but the mechanism is very complicated and has not been sufficiently revealed.

### CITATION LIST

### Patent Literature

Patent Literature 1: International Publication WO2008/001497
Patent Literature 2: JP Patent Publication No. 2014-094001 A
Patent Literature 3: International Publication WO2007/052081
Patent Literature 4: International Publication WO2011/049205

### Non Patent Literature

Non Patent Literature 1: Fumitaka Hayase, “Browning”, Journal of the Japanese Society of Food Science and Technology, 36 (1), p. 89-90 (1989)

### SUMMARY OF INVENTION

### Technical Problem

The problem underlying the present invention is to provide a fermentation promoting agent for Streptococcus thermophilus, that is suitable for use in food production.

### Solution to Problem

The present inventors conducted intensive studies to solve the above-mentioned problem, and have found that the fermentation of Streptococcus thermophilus can be promoted effectively by a colored solution obtained by exposing an alkaline solution containing a reducing sugar to a temperature in a certain range to induce a color reaction of sugar, whereby the present invention was accomplished.

More specifically, the present invention encompasses the followings.

[1] A fermentation promoting agent for Streptococcus thermophilus, comprising a solution prepared by exposing an alkaline solution comprising a reducing sugar to a temperature in the range of 5° C. or more and 135° C. or less to induce a color reaction of sugar.
[2] The fermentation promoting agent according to the above [1], wherein said temperature is 35° C. or more.
[3] The fermentation promoting agent according to the above [1] or [2], wherein said solution is prepared by heating the alkaline solution comprising a reducing sugar at 35 to 100° C.
[4] The fermentation promoting agent according to any one of the above [1] to [3], wherein the alkaline solution comprising a reducing sugar comprises 0.05 to 80 wt % of the reducing sugar.
[5] The fermentation promoting agent according to any one of the above [1] to [4], wherein the reducing sugar is at least one selected from the group consisting of glucose, galactose, fructose, arabinose, rhamnose, xylose, lactose, lactulose, galactooligosaccharide and dextrin.
[6] The fermentation promoting agent according to any one of the above [1] to [5], wherein the alkaline solution comprising a reducing sugar comprises 0.05 to 30 wt % of a hydroxide.
[7] The fermentation promoting agent according to any one of the above [1] to [6], wherein the alkaline solution comprising a reducing sugar comprises at least one of sodium hydroxide and potassium hydroxide as the hydroxide.
[8] The fermentation promoting agent according to the above [7], wherein the reducing sugar is lactose and the hydroxide is sodium hydroxide or potassium hydroxide.
[9] The fermentation promoting agent according to any one of according to any one of the above [1] to [8], wherein the alkaline solution comprising a reducing sugar comprises a food material comprising a reducing sugar.
[10] The fermentation promoting agent according to the above [9], wherein said food material is at least one of a fruit juice and a reconstituted skim milk.
[11] A method for producing a fermentation promoting agent for Streptococcus thermophilus, comprising exposing an alkaline solution comprising a reducing sugar to a temperature in the range of 5° C. or more and 135° C. or less to induce a color reaction of sugar, thereby preparing a solution having a fermentation promoting effect on Streptococcus thermophilus.
[12] The method according to the above [11], wherein said temperature is 35° C. or more.
[13] The method according to the above [11] or [12], wherein said solution is prepared by heating the alkaline solution comprising a reducing sugar at 35 to 100° C.
[14] The method according to any one of the above [11] to [13], wherein the alkaline solution comprising a reducing sugar comprises 0.05 to 80 wt % of the reducing sugar.
[15] The method according to any one of the above [11] to [14], wherein the reducing sugar is at least one selected from the group consisting of glucose, galactose, fructose, arabinose, rhamnose, xylose, lactose, lactulose, galactooligosaccharide and dextrin.
[16] The method according to any one of the above [11] to [15], wherein the alkaline solution comprising a reducing sugar comprises 0.05 to 30 wt % of a hydroxide.
[17] The method according to any one of the above [11] to [16], wherein the alkaline solution comprising a reducing sugar comprises at least one of sodium hydroxide and potassium hydroxide as the hydroxide.
[18] The method according to the above [17], wherein the reducing sugar is lactose and the hydroxide is sodium hydroxide or potassium hydroxide.
[19] The method according to any one of the above [11] to [18], wherein the alkaline solution comprising a reducing sugar comprises a food material comprising a reducing sugar.
[20] The method according to the above [19], wherein said food material is at least one of a fruit juice and a reconstituted skim milk.
[21] A method for promoting Streptococcus thermophilus fermentation, comprising adding the fermentation promoting agent according to any one of the above [1] to [10], to a fermentation substrate and culturing Streptococcus thermophilus in/on the fermentation substrate to ferment the fermentation substrate.
[22] The method according to the above [21], wherein Streptococcus thermophilus and Lactobacillus bulgaricus are mixed-cultured in/on the fermentation substrate.
[23] A method for producing a milk-fermented food, comprising fermenting a fermentation substrate comprising milk or a milk-derived product with the method according to the above [21] or [22].
[24] The method according to the above [23], wherein the milk-fermented product is fermented milk.

The disclosure of JP Patent Application No. 2016-120216, to which the present application claims the priority, are incorporated into the present specification.

### Advantageous Effects of Invention

According to the present invention, the fermentation of Streptococcus thermophilus can be promoted even with a small amount of fermentation promoting agent added.

### BRIEF DESCRIPTION OF DRAWINGS

FIG. 1 is a photograph showing the influence of temperature on the color tone of a 50% lactose solution prepared with a 25% NaOH solution. It shows, in order from the leftmost tube, samples kept at −20° C., 5° C., 25° C. and 37° C. and a sample heat-treated at 95° C.

FIG. 2 shows the influence of temperature on the fermentation promoting effect on S. thermophilus of a lactose-NaOH solution (addition rate of 0.0025% (vol/wt)). Solid square, −20° C.; solid triangle, 5° C.; solid diamond, 25° C.; x, 37° C.; open square, 95° C.; and open circle, control. The unit H of the fermentation time on the horizontal axis denotes time [hour(s)] (the same applies to the following figures).

FIG. 3 shows the influence of temperature on the fermentation promoting effect on S. thermophilus of a lactose-NaOH solution (addition rate of 0.0125% (vol/wt)). Solid square, −20° C.; solid triangle, 5° C.; solid diamond, 25° C.; and open circle, control.

FIG. 4 shows the fermentation promoting effect on S. thermophilus of a 0.1% lactose solution prepared with a 0.1% NaOH solution (addition rate of a lactose-NaOH solution: 1%). Solid square, an unheated lactose-NaOH solution; solid triangle, a heated lactose-NaOH solution; and open circle, control.

FIG. 5 shows the fermentation promoting effect on S. thermophilus of a 0.1% lactose solution prepared with a 0.1% NaOH solution (addition rate of a lactose-NaOH solution: 10%). Solid square, an unheated lactose-NaOH solution; solid triangle, a heated lactose-NaOH solution; and open circle, control.

FIG. 6 shows the influence of the concentration of a NaOH solution on the fermentation promoting effect. Solid square, 0% NaOH; solid triangle, 0.8% NaOH; solid diamond, 1.6% NaOH; solid circle, 8% NaOH; x, 27% NaOH; and open circle, control.

FIG. 7 shows the influence of the sugar concentration and the concentration of a NaOH solution on the fermentation promoting effect. Solid square, 25% lactose/27% NaOH; solid triangle, 50% lactose/27% NaOH; solid diamond, 70% lactose/40% NaOH; and open circle, control.

FIG. 8 shows the influence of addition rate of heat-treated “70% Lac/40% NaOH”, on the fermentation promoting effect on S. thermophilus. Solid square, 0.0005%; solid triangle, 0.00075%; solid diamond, 0.001%; x, 0.00125%; and open circle, control.

FIG. 9 shows the fermentation promoting effect on S. thermophilus of a lactose-KOH solution. Solid square, lactose-KOH solution; and open circle, control.

FIG. 10 shows the fermentation promoting effect on S. thermophilus of a sugar-alkaline solution prepared using each of various sugars. Solid square, glucose; solid triangle, galactose; solid diamond, fructose; solid circle, arabinose; x, rhamnose; open square, xylose; open triangle, xylitol; and open circle, control.

FIG. 11 shows the fermentation promoting effect on S. thermophilus of a sugar-alkaline solution prepared using each of various sugars. Solid square, lactulose; solid triangle, sucrose; solid diamond, trehalose; solid circle, dextrin; and open circle, control.

FIG. 12 shows the fermentation promoting effect on S. thermophilus of a sugar-alkaline solution prepared using each of various sugars. Solid square, sorbitol; solid triangle, mannitol; solid diamond, fructooligosaccharide; solid circle, galactooligosaccharide; and open circle, control.

FIG. 13 is a photograph showing the color tone after heating of a sugar-alkaline solution prepared using each of various sugars. A, sugar-alkaline solutions prepared with glucose, galactose, fructose, xylose, arabinose and rhamnose, in order from the left. B, sugar-alkaline solutions prepared with xylitol, mannitol and sorbitol, in order from the left.

FIG. 14 is a photograph showing the color tone after heating of a sugar-alkaline solution prepared using each of various sugars.

FIG. 15 shows the fermentation promoting effect on S. thermophilus of a solution prepared by adding NaOH to 100% fruit juice of grape and heating it. Solid square, grape juice; solid triangle, grape juice+10% NaOH; and open circle, control.

FIG. 16 shows the fermentation promoting effect on S. thermophilus of a solution prepared by adding NaOH to 100% fruit juice of grapefruit and heating it. Solid square, grapefruit juice; solid triangle, grapefruit juice+10% NaOH; and open circle, control.

FIG. 17 shows the fermentation promoting effect on S. thermophilus of a solution prepared by adding NaOH to 100% fruit juice of orange and heating it. Solid square, orange juice; solid triangle, orange juice+10% NaOH; and open circle, control.

FIG. 18 shows the fermentation promoting effect on S. thermophilus of a solution prepared by adding NaOH to 100% fruit juice of apple and heating it. Solid square, apple juice; solid triangle, apple juice+10% NaOH; and open circle, control.

FIG. 19 is a photograph showing the color tone after heating of a solution prepared by adding NaOH to a fruit juice or a reconstituted skim milk (SMP).

FIG. 20 shows the fermentation promoting effect on S. thermophilus of a sugar-alkaline solution prepared with a reconstituted skim milk (SMP). Solid square, 10% SMP added; solid triangle, 10% SMP+5% NaOH added; and open circle, control.

FIG. 21 shows the fermentation promoting effect of a sugar-alkaline solution on S. thermophiles OLS3059 strain.

FIG. 22 shows the fermentation promoting effect of a sugar-alkaline solution on S. thermophiles OLS3294 strain.

FIG. 23 shows the fermentation promoting effect of a sugar-alkaline solution on S. thermophiles OLS3289 strain.

FIG. 24 shows the fermentation promoting effect of a sugar-alkaline solution on S. thermophiles OLS3469 strain.

FIG. 25 shows the fermentation promoting effect of a sugar-alkaline solution on S. thermophiles OLS3058 strain.

FIG. 26 shows the fermentation promoting effect of a sugar-alkaline solution on S. thermophiles OLS3290 strain.

FIG. 27 shows the fermentation promoting effect, with an acidity as the indicator, of a sugar-alkaline solution in mixed fermentation of S. thermophilus and L. bulgaricus.

FIG. 28 shows, with L-lactic acid concentration as an indicator, the fermentation promoting effect of a sugar-alkaline solution in mixed fermentation of S. thermophilus and L. bulgaricus.

### DESCRIPTION OF EMBODIMENTS

Hereinafter, the present invention will be described in detail.

The present invention is based on the present inventors' finding that when an alkaline solution containing a reducing sugar (hereinafter sometimes referred to as “sugar-alkaline solution”) causes a color reaction under a temperature condition of 5° C. or more, the resulting solution has an action of promoting Streptococcus thermophilus fermentation.

The present invention relates to a fermentation promoting agent for Streptococcus thermophilus, comprising a solution prepared by exposing an alkaline solution comprising a reducing sugar to a temperature typically in the range of 5° C. or more and 135° C. or less to induce a color reaction of sugar.

The term “reducing sugar” as used herein refers to a sugar which generates an aldehyde group or a ketone group (a reducing end) in a basic solution. In the present invention, a reducing sugar may be selected from monosaccharides, disaccharides, oligosaccharides (those having an average polymerization degree of 3 to 30, in the present invention), or polysaccharides (having an average polymerization degree of 31 or more, for example, 31 to 1000) or any combination thereof. The reducing sugar which is a monosaccharide may be a hexose (aldohexose or ketohexose) or an aldose. Preferred examples of monosaccharide reducing sugar include, but are not limited to, e.g., glucose, galactose, fructose, arabinose, rhamnose and xylose. Preferred examples of disaccharide reducing sugar include, but are not limited to, e.g., lactose, lactulose and maltose. Preferred examples of oligosaccharide reducing sugar include, but are not limited to, e.g., galactooligosaccharide, xylooligosaccharide and isomaltooligosaccharide. Preferred examples of polysaccharide reducing sugar include, but are not limited to, e.g., dextrin. The solution containing a reducing sugar and an alkali according to the present invention may contain one or more reducing sugars.

According to the present invention, a food material containing a reducing sugar may be used for preparing a sugar-alkaline solution. That is, the sugar-alkaline solution according to the present invention may contain one or more food materials containing a reducing sugar, and also in this case, such solution “contains a reducing sugar”. The term “food material” as used herein means a material used for food production, which may or may not be used as a food or food additive by itself. The food material containing a reducing sugar may be in any form such as a liquid, a semi-liquid or a solid (a powder, a granule or the like) but it is preferably soluble in an aqueous solution. Examples of the food material containing a reducing sugar include, but are not limited to, a fruit juice, a vegetable juice, a reconstituted skim milk, milk, whey (milk serum), a whey protein concentrate (WPC), a whey filtrate, other dairy materials, a reducing sugar-containing beverage, fermented milk, high fructose-corn syrup (HFCS; or glucose-fructose syrup), honey, a fruit or vegetable extract (an aqueous extract). Among these, dairy materials such as whey (milk serum), a whey protein concentrate and a whey filtrate contain lactose, which is a reducing sugar, at a high concentration and are preferably used in the present invention. The term “fruit juice” as used herein includes squeezed juice of a fruit and processed products thereof (such as concentrates, products reconstituted from concentrate, and dilutions, and sweetened products thereof). Fruit juice is generally rich in a reducing sugar such as fructose or glucose. Examples of the fruit juice include, but are not limited to, juices of citrus fruits such as orange, grapefruit or unshu mikan (Citrus unshiu); grape juice, apple juice, mango juice, peach juice, pineapple juice, strawberry juice, pear juice, lemon juice, banana juice and melon juice. The fruit juice may be also mixed juice of two or more fruit juices. Examples of the food materials containing a reducing sugar also include a mixed juice of a fruit juice and a vegetable juice. The “vegetable juice” includes squeezed juice of a vegetable (such as tomato and carrot) and processed products thereof (such as concentrates, products reconstituted from concentrate, and dilutions, and sweetened products thereof). In one embodiment, the sugar-alkaline solution of the present invention may contain at least one of a fruit juice, a reconstituted skim milk, whey (milk serum), a whey protein concentrate and a whey filtrate.

The sugar-alkaline solution of the present invention usually comprises 0.05 wt % or more, preferably 0.05% to 80 wt %, for example, 5 wt % to 75 wt % or 10 wt % to 70 wt % of a reducing sugar, relative to the total weight of the solution. In one embodiment, the sugar-alkaline solution of the present invention also preferably comprises a reducing sugar at a high concentration, for example, in an amount of 20 wt % or more or 50 wt % or more relative to the total weight of the solution. The reducing sugar concentration herein means a final concentration after preparing the solution. The unit wt % (weight percent; w/w %) relative to the total weight may be also denoted by “% (wt/wt)” or “wt/wt (%)”.

The term “alkaline solution” as used herein refers to an aqueous solution having a hydroxide (hydroxide salt) dissolved therein (e.g., an aqueous solution of a hydroxide). An alkaline solution can be prepared by adding a hydroxide to an aqueous solution and dissolving it therein. Accordingly, the sugar-alkaline solution of the present invention contains a reducing sugar and a hydroxide. The hydroxide to be used for preparing the sugar-alkaline solution of the present invention is preferably a hydroxide that can be used for food production, and it may be an alkali metal hydroxide and is typically sodium hydroxide or potassium hydroxide. The sugar-alkaline solution of the present invention preferably comprises at least one of sodium hydroxide and potassium hydroxide dissolved therein.

The sugar-alkaline solution of the present invention comprises a hydroxide, and specifically it may usually contain 0.05 wt % or more, preferably 40 wt % or less, more preferably 0.05 wt % to 30 wt %, for example, 0.5 wt % or more, 0.5 wt % to 30 wt %, 5 wt % to 25 wt % or 10 wt % to 20 wt % of a hydroxide, relative to the total weight of the solution. In one embodiment, the sugar-alkaline solution of the present invention may contain a hydroxide at a high concentration, for example, in an amount of 20 wt % or more, relative to the total weight of the solution. The hydroxide concentration herein means a final concentration after preparing the sugar-alkaline solution.

In one embodiment, the sugar-alkaline solution of the present invention contains lactose as a reducing sugar and sodium hydroxide or potassium hydroxide as a hydroxide. In this case, the hydroxide concentration is as described above, and may be 0.05 to 30 wt %, for example. The reducing sugar concentration is also as described above, and may be 0.05 to 80 wt %, for example.

The sugar-alkaline solution of the present invention can be prepared by a conventional method. The sugar-alkaline solution of the present invention may be, for example, prepared by adding a reducing sugar or a food material containing a reducing sugar to an alkaline solution and dissolving it therein. The sugar-alkaline solution of the present invention can also be prepared by dissolving a hydroxide in an aqueous solution containing a reducing sugar or a liquid food material containing a reducing sugar. Alternatively, the sugar-alkaline solution of the present invention can also be prepared by dissolving a reducing sugar or a food material containing a reducing sugar and a hydroxide in an aqueous solution. The sugar-alkaline solution of the present invention may be prepared under a temperature condition of less than 5° C. or under a temperature condition of 5° C. or more, for example, at ordinary temperature (20 to 25° C.). The term “solution” as used herein means a liquid in which a solute looks like to be uniformly dispersed in a solvent by visual observation, and it encompasses a liquid in which a solute is dispersed as monomeric molecules in a solvent, and a liquid in which an aggregate or colloidal particle of a solute is dispersed in a solvent is dispersed in a solvent (colloid or the like). The term “solution” as used herein also encompasses such a liquid in which a solute is uniformly dispersed in a solvent, but some amount of solute or insoluble ingredient remains undissolved and is present as a precipitate or the like in the liquid.

The sugar-alkaline solution of the present invention may be prepared by adding a reducing sugar to an alkaline solution (typically 0.05 wt % or more, preferably 0.5 to 50 wt %, more preferably 1 to 40 wt %, for example, 5 to 20 wt % or 20 to 50 wt % alkaline solution) and dissolving the reducing sugar therein. In the present invention, it is also preferred to use, as a sugar-alkaline solution, the thus obtained solution containing 0.05 wt % to 80 wt %, for example, 10 wt % to 70 wt %, of the reducing sugar.

The sugar-alkaline solution of the present invention may contain other ingredients in addition to water, a reducing sugar and a hydroxide. For example, when the sugar-alkaline solution of the present invention is prepared using a food material containing a reducing sugar, ingredients other than the reducing sugar contained in the food material are present in the sugar-alkaline solution of the present invention. However, the sugar-alkaline solution of the present invention need not contain an amino compound (an amino acid, a peptide and a protein) for color reaction, and it may not contain an amino compound in an amount that can induce coloring by Maillard reaction, or may contain no amino compound. The sugar-alkaline solution of the present invention also does not contain an alkaline copper reagent for quantification of sugar.

According to the present invention, the sugar-alkaline solution as described above is exposed to a temperature of preferably 5° C. or more, typically in the range of 5° C. or more and 135° C. or less (in one embodiment, 20° C. or more, preferably 35° C. or more, more preferably 50° C. or more and further preferably 80° C. or more, and/or 100° C. or less, preferably 99° C. or less and more preferably 98° C. or less) to induce a color reaction. In one preferred embodiment of the present invention, the temperature to which the sugar-alkaline solution is exposed may be typically of 5° C. to 135° C., and additionally or alternatively, may be a temperature below the melting point of the sugar to be used in the solution (for example, a temperature by 5° C. or more lower than the lower limit of the melting point). The expression “exposing a sugar-alkaline solution to a temperature in the range of 5° C. or more and 135° C. or less” means that the temperature of the solution is kept at a given temperature or within a given temperature range in the range of 5° C. or more and 135° C. or less for a certain period of time by refrigeration, incubation, storage or the like, or the solution is heat-treated at a given temperature in the range of 5° C. or more and 135° C. or less for a certain period of time. Exposure to such temperature may be carried out, for example, by heating the sugar-alkaline solution at 35 to 100° C. The expression “exposing a sugar-alkaline solution to a temperature lower than the melting point of the sugar used in the solution” is interpreted similarly except for using a temperature lower than the melting point.

When exposing the sugar-alkaline solution of the present invention to a temperature in the range of 5° C. or more, a color reaction of sugar is induced and also promoted by heat of dissolution of the sugar (the heat generated during dissolution of the sugar in a liquid) and/or the artificially applied heat. The color reaction can be induced by keeping the sugar-alkaline solution of the present invention at 5° C. or more, preferably 5° C. to 50° C. and more preferably 20° C. or more, for example 35° C. to 40° C., for a certain period of time, for example, 10 minutes to 24 hours, preferably 1 hour to 12 hours and more preferably 3 to 6 hours. The term “keeping” as used herein includes not only keeping the sugar-alkaline solution at the same temperature but also allowing the temperature of the sugar-alkaline solution to fall within a certain temperature range (for example, of 35° C. to 40° C.). In order to keep the temperature of the sugar-alkaline solution of the present invention, for example, the sugar-alkaline solution of the present invention in a container may be refrigerated or stored at ordinary temperature or room temperature, or it may be incubated using a warmer such as an incubator. The sugar-alkaline solution of the present invention can be also heat-treated at 30° C. or more, typically in the range of 35° C. or more and 135° C. or less (that is, 35° C. to 135° C.), preferably 35° C. to 100° C., more preferably 50 to 100° C., e.g., 80° C. or more and/or 99° C. or less and further preferably 70 to 98° C., for a certain period of time, for example, 10 minutes or more, preferably 20 minutes to 2 hours and more preferably 20 minutes to 1 hour, to induce a color reaction more rapidly. The sugar-alkaline solution of the present invention may be kept at a temperature of 5° C. or more, for example, 5° C. to 35° C. for a certain period of time, followed by heating at the above-mentioned temperature, or may be heated preferably at a temperature of 35 to 135° C., for example, 35 to 100° C. The expression “heating a sugar-alkaline solution” at the above-mentioned temperature means that heat is applied to the sugar-alkaline solution so that the solution reaches the above-mentioned temperature. The expression “induce a color reaction of sugar” as used herein means that a color reaction of sugar is caused and as a result, a colorized sugar-alkaline solution is produced. Specifically, via the color reaction of sugar, the sugar-alkaline solution turns from colorless or another color to brown to black or turns deeper brown to black than the original color of the solution (browning/blackening). The color reaction of sugar in the sugar-alkaline solution can be promoted by increasing the sugar concentration and/or the hydroxide concentration in the sugar-alkaline solution; or in addition to or alternatively to, by increasing the temperature at which and/or the period of time for which the sugar-alkaline solution is heated or kept. The sugar-alkaline solution may be heated after a color react is started by, e.g., heat of dissolution in the sugar-alkaline solution.

In this manner, a brown- to black-colorized solution (colored liquid) can be prepared from the sugar-alkaline solution. This colored liquid has an action of markedly promoting Streptococcus thermophilus fermentation and can be used for promoting fermentation on Streptococcus thermophilus. The present invention provides a fermentation promoting agent for Streptococcus thermophilus comprising a colored liquid prepared as described above (hereinafter referred to as a fermentation promoting liquid). This fermentation promoting liquid may be a composition comprising a reducing sugar, a hydroxide, water and a product generated in association with a color reaction, and optionally an ingredient derived from a food material containing a reducing sugar, and the like.

Streptococcus thermophilus is cultured in/on a fermentation substrate supplemented with the fermentation promoting liquid or fermentation promoting agent of the present invention, and an indicator of progress of the fermentation is examined over time. As a result, if it is shown that the fermentation has proceeded faster than in a control (that is a group without fermentation promoting liquid/fermentation promoting agent), the fermentation promoting liquid or fermentation promoting agent can be verified to have a fermentation promoting effect. As the indicator of progress of fermentation, for example, an increase in the amount of L-lactic acid produced by Streptococcus thermophilus fermentation, or an increase in the acidity or a decrease in pH value of a fermented product associated with an increase in the amount of L-lactic acid, can be used, but the indicator is not limited thereto. If the value of the indicator of progress of fermentation is improved as compared with the control, the difference in the value of the indicator from the control enlarges with time during fermentation (preferably for at least 2 hours), and then an improved value of the indicator is still shown as compared with the control for a certain period of time (for example, for at least 1 hour or more), the fermentation promoting liquid or fermentation promoting agent can be determined to have a fermentation promoting effect on Streptococcus thermophilus. The acidity (weight percent concentration of lactic acid) of a fermentation product can be determined, for example, by gradually adding dropwise phenolphthalein to the fermentation product, determining the amount of 0.1N NaOH (=0.1 mol/L NaOH) required to turn pale red (about pH 8.5), and calculating the acidity therefrom in a conventional manner. Further, an L-lactic acid concentration can be measured, for example, with high performance liquid chromatography (HPLC) at the temperature of 40° C. using a mobile phase of 2 mM CuSO₄(II).5H₂O and 5% 2-propanol. For the specific test procedures, the descriptions of Examples below can be referred to.

The fermentation promoting liquid of the present invention can also promote the growth of Streptococcus thermophilus. Therefore, the fermentation promoting liquid or fermentation promoting agent of the present invention can be also used as a growth promoting agent for Streptococcus thermophilus. The present invention also provides a growth promoting agent for Streptococcus thermophilus comprising the fermentation promoting liquid or fermentation promoting agent of the present invention.

The fermentation promoting liquid of the present invention can be used as an active ingredient of a fermentation promoting agent for Streptococcus thermophilus of the present invention. The fermentation promoting liquid of the present invention may be used as it is in the form of a colored liquid prepared as described above as an active ingredient of the fermentation promoting agent for Streptococcus thermophilus of the present invention. Alternatively, the fermentation promoting liquid of the present invention may be used as an active ingredient of the fermentation promoting agent for Streptococcus thermophilus after subjecting it to a treatment such as concentration, dilution, filtration, sterilization, homogenization, drying, gelling, granulation and/or powderization. These treatments usually do not irreversibly inactivate the fermentation promoting action. The fermentation promoting agent for Streptococcus thermophilus according to the present invention encompasses not only a formulation using the prepared colored liquid directly but also a formulation containing one obtained by subjecting the prepared colored liquid to such a treatment.

The fermentation promoting agent or growth promoting agent for Streptococcus thermophilus according to the present invention may further comprise other ingredients, typically an auxiliary substance used in the field of production of foods or food additives, such as a carrier, an excipient, or a preservative. The fermentation promoting agent or growth promoting agent for Streptococcus thermophilus according to the present invention may be a composition further containing such other ingredients. The fermentation promoting agent for Streptococcus thermophilus may be a liquid or in any other form such as a powder, a granule, a gel, a solid or an encapsulated form. The powderization, granulation, gelling, solidification, encapsulation and the like can be carried out in accordance with known formulation techniques.

As described above, the present invention also provides a method for producing the above-mentioned fermentation promoting agent for Streptococcus thermophilus, comprising exposing an alkaline solution comprising a reducing sugar to a temperature of 5° C. or more, typically in the range of 5° C. or more and 135° C. or less to induce a color reaction of sugar, thereby preparing a solution having a fermentation promoting effect on Streptococcus thermophilus (fermentation promoting liquid). Various conditions for this production method such as types and concentrations of reducing sugar and hydroxide used, the temperature to which the alkaline solution comprising the reducing sugar is exposed, and the composition and the preparation method of the alkaline solution comprising a reducing sugar are as described above. This production method may comprise a step of formulating, as an active ingredient, the above fermentation promoting liquid having a fermentation promoting effect on Streptococcus thermophilus into a fermentation promoting agent. This production method may comprise treating the fermentation promoting liquid by e.g., concentration, dilution, filtration, sterilization, homogenization, drying, gelling, granulation and/or powderization. These treatments usually do not irreversibly inactivate the fermentation promoting action.

The present invention also provides a method for promoting fermentation by Streptococcus thermophilus using the fermentation promoting agent of the present invention. More specifically, the present invention also provides a method for promoting Streptococcus thermophilus fermentation, comprising adding the fermentation promoting agent of the present invention to a fermentation substrate and culturing Streptococcus thermophilus in/on the fermentation substrate to ferment the fermentation substrate. The present invention also relates to a fermentation method using Streptococcus thermophilus, comprising adding the fermentation promoting agent of the present invention to a fermentation substrate and culturing Streptococcus thermophilus in/on the fermentation substrate. The present invention also relates to a method for producing a lactic acid bacterial product, comprising adding the fermentation promoting agent of the present invention to a fermentation substrate, culturing Streptococcus thermophilus in/on the fermentation substrate, and collecting a lactic acid bacterial product produced by Streptococcus thermophilus. The present invention further provides a method for growing Streptococcus thermophilus, comprising promoting the growth of Streptococcus thermophilus by using the fermentation promoting agent of the present invention. In these methods, Streptococcus thermophilus may be inoculated into the fermentation substrate before adding the fermentation promoting agent of the present invention to the fermentation substrate or at the same time as or after adding the fermentation promoting agent to the fermentation substrate.

The term “fermentation substrate” as used herein means a substrate compound (such as a carbohydrate) or a substrate material that is available for fermentation by Streptococcus thermophilus. Examples of the fermentation substrate include, but are not limited to, milk, a milk-derived product, a saccharified cereal, soymilk, a soybean extract, a fruit, a vegetable, a fruit juice, a vegetable juice, a fruit or vegetable extract, and a fermentation broth base (for example, a yogurt base) containing at least one thereof. The term “milk” as used herein includes raw milk, raw milk after composition adjustment (composition standardization), fat-reduced or non-fat milk (such as skim milk); a powdered milk such as a powdered skim milk and whole milk powder; a reconstituted skim milk, a diluted milk, a concentrated milk, and other processed milks. The “milk” may be subjected to a pretreatment used in food production such as homogenization, sterilization/cooling, and/or filtration. In the context of the present invention, the “milk” may be any non-human mammal milk (animal milk) such as cow milk, goat milk, buffalo milk, horse milk, camel milk or sheep milk. The “milk-derived product” may or may not contain lactose, but preferably contains lactose. Examples of the “milk-derived product” include a curd (coagulated milk), a cream, buttermilk, a buttermilk powder, whey, milk protein (such as casein or whey protein) and a hydrolysate thereof (such as casein-hydrolyzed peptide). The fermentation substrates may be used alone or in combination of two or more in the present invention.

The fermentation promoting liquid or fermentation promoting agent of the present invention, in principle, exhibits a higher fermentation promoting effect as the sugar concentration and the hydroxide concentration used in preparing the fermentation promoting liquid is higher. Therefore, the amount of the fermentation promoting liquid or fermentation promoting agent of the present invention required to be added for promoting Streptococcus thermophilus fermentation can be lower as the sugar concentration and the hydroxide concentration used in preparing the fermentation promoting liquid is higher, and the specific amount to be added can be appropriately adjusted by those skilled in the art. The fermentation promoting agent of the present invention may be generally added in an amount such that the fermentation promoting liquid is added at 0.0001% (vol/wt) or more and preferably 20% (vol/wt) or less, and more preferably 0.0005 to 10% (vol/wt), for example, 0.001 to 1% (vol/wt) or 0.01 to 5% (vol/wt), relative to the total weight of the fermentation substrate(s). Herein, % (vol/wt) represents percentage (%) of the volume (ml) of the fermentation promoting liquid relative to the total weight (g). Thus, the fermentation promoting liquid or fermentation promoting agent of the present invention can promote the fermentation of Streptococcus thermophilus even by adding it in a very small amount. This means not only that the production cost of a fermented food can be reduced but also that the influence on the taste of the fermented food can be markedly reduced or prevented.

The fermentation promoting agent of the present invention can be used for any strain of Streptococcus thermophilus. Examples of the strain of Streptococcus thermophilus include, but are not limited to, S. thermophilus OLS3059 strain (accession number FERM BP-10740), S. thermophilus OLS3294 strain (accession number NITE P-77), S. thermophilus OLS3289 strain (ATCC 19258), S. thermophilus OLS3469 strain (IFO 13957/NBRC 13957), S. thermophilus OLS3058 strain, and S. thermophilus OLS3290 strain (accession number FERM BP-19638).

S. thermophilus OLS3059 strain is internationally deposited under the Budapest Treaty with International Patent Organism Depositary, National Institute of Technology and Evaluation (NITE-IPOD) (#120, 2-5-8, Kazusakamatari, Kisarazu-shi, Chiba, Japan) on Feb. 29, 1996 (the date of the original deposit) under the accession number FERM BP-10740. This deposited strain was transferred from the domestic deposit (the original deposit) to the international deposit under the Budapest Treaty on Nov. 29, 2006.

S. thermophilus OLS3294 strain is deposited with Patent Microorganisms Depositary, National Institute of Technology and Evaluation (NPMD) (#122, 2-5-8, Kazusakamatari, Kisarazu-shi, Chiba, Japan) on Feb. 10, 2005 (the date of deposit) under the accession number NITE P-77.

Further, S. thermophilus OLS3290 strain is internationally deposited under the Budapest Treaty with International Patent Organism Depositary, National Institute of Technology and Evaluation (NITE-IPOD) (#120, 2-5-8, Kazusakamatari, Kisarazu-shi, Chiba, Japan) on Jan. 19, 2004 (the date of the original deposit) under the accession number FERM BP-19638. This deposited strain was transferred from the domestic deposit (the original deposit) to the international deposit under the Budapest Treaty on Sep. 30, 2013.

S. thermophilus OLS3289 strain is the same as the bacterium that is available under ATCC® Catalog No. 19258 from the American Type Culture Collection (ATCC).

S. thermophilus OLS3469 strain is the same as the bacterium that is available under NBRC No. 13957 from Biological Resource Center, Biotechnology Center, National Institute of Technology and Evaluation (NBRC) (2-5-8, Kazusakamatari, Kisarazu-shi, Chiba, Japan).

Note that Meiji Co., Ltd. is not only the depositor of S. thermophilus OLS3290 strain, but is also the current depositor of S. thermophilus OLS3059 strain and S. thermophilus OLS3294 strain.

The fermentation (culture) conditions for Streptococcus thermophilus can be set according to a conventional method. For example, fermentation can usually be carried out at 35 to 50° C. and preferably at 40 to 45° C. The fermentation time varies depending on the fermentation substrate and fermentation conditions, but it can be set to, for example, about 2 to 24 hours. If necessary, the pH of the fermentation substrate may be appropriately adjusted (for example, adjusted to around pH 6.5) before fermentation.

Streptococcus thermophilus can be prepared according to a conventional method. Streptococcus thermophilus may be inoculated in any amount that can be used for fermentation by Streptococcus thermophilus. For example, the inoculation amount of Streptococcus thermophilus can be set in the range of 0.01 to 5% (v/w %) expressed as a ratio of inoculation amount (ml) to the total weight (g) of the fermentation substrate. The % ratio of the volume to the total weight (v/w %) may be also denoted as % (vol/wt) or vol/wt (%). Since the fermentation promoting liquid or fermentation promoting agent of the present invention can markedly promote Streptococcus thermophilus fermentation, the inoculation amount of Streptococcus thermophilus can be reduced, for example, to approximately 1/10 to ⅔ of the typical inoculation amount (the number of bacterial cells to be inoculated).

In the method of the present invention, it is also preferred to mixed-culture (co-culture) Streptococcus thermophilus and Lactobacillus bulgaricus. The fermentation promoting liquid or fermentation promoting agent of the present invention can also promote Streptococcus thermophilus fermentation in the mixed culture of Streptococcus thermophilus and Lactobacillus bulgaricus. In a preferred embodiment, the mixed culture of Streptococcus thermophilus and Lactobacillus bulgaricus, is carried out by using a fermentation substrate comprising milk or a milk-derived product.

In the typical yogurt production, mixed-culture (mixed fermentation) of Streptococcus thermophilus and Lactobacillus bulgaricus is carried out. Streptococcus thermophilus is also often used in the production of fermented foods including various cheeses such as mozzarella cheese. The method for promoting Streptococcus thermophilus fermentation according to the present invention is also very useful for producing a fermented food more efficiently. The present invention also provides a method for producing a fermented food comprising fermenting a fermentation substrate by the method for promoting Streptococcus thermophilus fermentation according to the present invention. Streptococcus thermophilus is generally used as a starter in the production of a fermented food. The fermentation substrate used for a fermented food is preferably edible itself (for example, for human or non-human mammals such as domesticated animals). The fermentation substrate may be used alone or in combination of two or more in the method for producing a fermented food.

In a preferred embodiment, the present invention relates to a method for producing a milk-fermented food, comprising fermenting a fermentation substrate comprising milk or a milk-derived product by the method for promoting Streptococcus thermophilus fermentation according to the present invention. This fermentation of the fermentation substrate is carried out using Streptococcus thermophilus or microorganisms comprising Streptococcus thermophilus. The fermentation substrate comprising milk or a milk-derived product may be milk or a milk-derived product itself. The definitions of milk and milk-derived product are as described above. The fermentation substrate comprising milk or a milk-derived product may be milk or a milk-derived product supplemented with another substrate compound (such as carbohydrate) or a substrate material or another ingredient. Examples of the fermented food (milk-fermented food) produced by this method include, but are not limited to, fermented milk, lactic acid bacteria fermented product-containing beverage, cheese, fermented cream and fermented butter. The term “fermented milk” as used herein refers to milk fermented using a lactic acid bacterium or a combination of a lactic acid bacterium and another fermentation microorganism (typically, yeast). Examples of the fermented milk include yogurt. The term “yogurt” as used herein refers to milk fermented using Streptococcus thermophilus and a Lactobacillus bacterium (such as Lactobacillus bulgaricus). Examples of the cheese include mozzarella cheese, Camembert cheese, quark cheese, Gouda cheese and cheddar cheese. In this method for producing a milk-fermented food, the fermentation substrate may be used alone or in combination of two or more thereof. For example, fermentation substrates containing two or more types of milk, e.g., raw milk and a powdered skim milk, may be used. Alternatively, milk and a milk-derived product may be used in combination as a fermentation substrate, and for example, raw milk, a powdered skim milk and whey protein may be used in combination. Further, a fermentation substrate comprising milk or a milk-derived product and a fermentation substrate not comprising any milk or milk-derived product may be used in combination. A fermentation broth base wherein a required amount of water and/or another ingredient such as a sweetener are added to and mixed with such fermentation substrate, can also be used as a fermentation substrate.

The method for producing a milk-fermented food according to the present invention can be carried out essentially by the same method as the conventional method for producing a milk-fermented food, except that the fermentation promoting agent of the present invention is added in an appropriate amount to a fermentation system to promote fermentation of Streptococcus thermophilus. After completing the fermentation to achieve appropriate conditions for respective milk-fermented foods, the resulting fermented products may be e.g., processed and filled in containers to produce milk-fermented foods. For example, fermented milk can be produced by inoculating lactic acid bacteria comprising Streptococcus thermophilus into milk, to which the fermentation promoting agent of the present invention is added according to the above-mentioned fermentation promoting method, and fermenting the milk. Typical yogurt can be produced by inoculating Streptococcus thermophilus and Lactobacillus bacterium (typically Lactobacillus bulgaricus) into milk, to which the fermentation promoting agent of the present invention is added according to the above-mentioned fermentation promoting method, and fermenting the milk in a mixed culture thereof. However, procedures for producing fermented milk, including yogurt, are not limited thereto.

In the method for producing a fermented food according to the present invention, a lactic acid bacterium which is known to be used for producing a fermented food (for example, a milk-fermented food) can be preferably used together with Streptococcus thermophilus. Lactobacillus bulgaricus (or Lactobacillus delbrueckii subsp. bulgaricus) to be used can be any strain which can be used for producing a fermented food, and examples of the strain include, but are not limited to, Lactobacillus delbrueckii subsp. bulgaricus OLL1073R-1 strain (accession number FERM BP-10741), Lactobacillus bulgaricus OLL1181 strain (accession number FERM BP-11269) and L. bulgaricus OLL1255 strain (accession number NITE BP-76).

Lactobacillus bulgaricus OLL1073R-1 strain is internationally deposited under the Budapest Treaty with International Patent Organism Depositary, National Institute of Technology and Evaluation (NITE-IPOD) (#120, 2-5-8 Kazusakamatari, Kisarazu-shi, Chiba, Japan) under the accession number FERM BP-10741 (the date of the original deposit: Feb. 22, 1999). This strain was transferred from the domestic deposit (the original deposit) to the international deposit on Nov. 29, 2006.

Lactobacillus bulgaricus OLL1181 strain is internationally deposited under the Budapest Treaty with International Patent Organism Depositary, National Institute of Technology and Evaluation (NITE-IPOD) (#120, 2-5-8 Kazusakamatari, Kisarazu-shi, Chiba, Japan) under the accession number FERM BP-11269 (the date of the original deposit: Jul. 16, 2010).

Lactobacillus bulgaricus OLL1255 strain is internationally deposited under the Budapest Treaty with Patent Microorganisms Depositary, National Institute of Technology and Evaluation (NPMD) (#122, 2-5-8 Kazusakamatari, Kisarazu-shi, Chiba, Japan) under the accession number NITE BP-76 (the date of the original deposit: Feb. 10, 2005). This strain was transferred from the domestic deposit (the original deposit) to the international deposit on Apr. 1, 2009.

The current depositor of Lactobacillus bulgaricus OLL1073R-1 strain, Lactobacillus bulgaricus OLL1181 strain, and Lactobacillus bulgaricus OLL1255 strain is Meiji Co., Ltd.

Other material(s) in addition to milk may be added at an appropriate stage in the production of a milk-fermented food. Examples of the other materials include, but are not limited to, food additives such as a sweetener (sucrose, stevia, sucralose or the like), an acidifier, a preservative, a flavor, a thickener, and calcium lactate; agar, gelatin, fruit juice, fruit pulp, fruit sauce, cream, aloe mesophyll and jam. It is usually preferred not to add a yeast extract known as a bifidobacteria growth-promoting agent, in order to avoid increased unpleasant tastes.

The production of fermented milk such as yogurt may be carried out by either a pre-fermentation type method or a post-fermentation type method. In the pre-fermentation type method, milk is inoculated with lactic acid bacteria (starter) comprising Streptococcus thermophilus, and after completion of the fermentation, the resulting fermented milk is filled into a container. Homogenization, addition of other materials such as fruit pulp, freezing or the like may be carried out before filling into the container. In the post-fermentation type method, the fermentation is carried out after filling milk, lactic acid bacteria and other materials into a container. The mixed culture of Streptococcus thermophilus and a Lactobacillus bacterium such as Lactobacillus bulgaricus can usually be carried out usually at 35 to 50° C. and preferably at 40° C. to 45° C. In the production of fermented milk, the fermentation is usually carried out until the acidity reaches 0.7 to 0.8%, followed by cooling to 10° C. or less to stop the fermentation, but the production method is not limited to. The fermentation time can be, for example, 1 to 24 hours and more generally approximately 3 to 7 hours.

Cheese can be typically produced by inoculating lactic acid bacteria (starter) comprising Streptococcus thermophilus into milk, to which the fermentation promoting agent of the present invention is added according to the above-mentioned fermentation promoting method, and fermenting the milk; then adding rennet (milk-curdling enzymes) to curdle the milk; separating a curdled product (curd) from whey; and shaping, sterilizing and/or fermenting and aging it or the like. However, procedures for producing cheese are not limited thereto.

The fermentation time can be reduced by the method of the present invention as compared with the method without the fermentation promoting agent of the present invention, since the present method can significantly promote lactic acid bacteria fermentation. For example, when fermented milk such as yogurt is produced according to the present method, the fermentation time can be preferably reduced by 1 to 4 hours as compared with the method without the fermentation promoting agent of the present invention, but the time reduction is not limited thereto because it varies depending on fermentation conditions or the like. According to the present method, the fermentation process in the production of a milk-fermented food can be completed early, making the production of milk-fermented foods more efficient.

When using such method of the present invention, it is possible to produce a milk-fermented food which is comparable to or more excellent in tastes (sourness and sweetness, and the presence or absence of bitterness and harsh taste, and the like) and physical properties (such as smoothness and firmness), as compared with milk-fermented foods produced in the same manner except that the fermentation promoting agent of the present invention is not added.

### EXAMPLES

Hereinafter, the present invention is further specifically described by reference to Examples. However, the technical scope of the present invention is not limited to these Examples.

### [Example 1] Fermentation Promoting Effect of NaOH Solution Having Lactose Dissolved Therein

Lactose was dissolved in a 25% (wt/wt) NaOH solution (aqueous NaOH solution) to prepare a 50% (wt/wt) lactose solution (hereinafter, a sodium hydroxide (NaOH) solution having lactose dissolved therein was also referred to as “lactose-NaOH solution”). The dissolution of lactose was carried out in an ice-water bath. The resulting lactose-NaOH solution was a clear liquid which was somewhat tinged with yellowish green.

For the temperature-kept group, the resulting lactose-NaOH solution was kept at −20° C., 5° C., 25° C. or 37° C. for 4 hours. For the heated group, the resulting lactose-NaOH solution was heated at 95° C. for 30 minutes immediately after preparation and then stored at a low temperature. Thereafter, the observation of the appearance of the temperature-kept or heated solution was carried out.

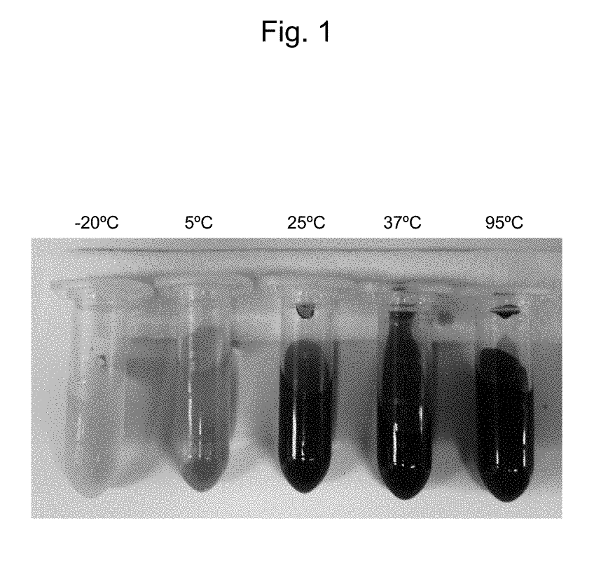

As a result, the solution kept at −20° C. exhibited no change in color. On the other hand, the solution kept at 5° C. exhibited some browning, the solution kept at 25° C. somewhat blackened and the solution kept at 37° C. blackened. The solution heated at 95° C. for 30 minutes blackened strongly. The photograph showing the color tone of each solution is shown in FIG. 1.

Each lactose-NaOH solution was added at 0.0025% (vol/wt) to UHT sterilized milk (cow milk sterilized by a UHT method (ultrahigh temperature sterilization method); sterilized at 130° C. for 2 seconds), and warmed to 43° C. Streptococcus thermophilus (S. thermophilus) OLS3059 strain (accession number FERM BP-10740) was inoculated as a starter in an amount of 1% (vol/wt) (1 to 2×10⁷ cfu/mL of bacterial cell concentration) into the warmed solution and fermentation was started at 43° C. As a control, fermentation was carried out by using UHT sterilized milk having sterilized water added thereto instead of the lactose-NaOH solution. For S. thermophilus OLS3059 strain, the bacterial cells obtained by culturing at 37° C. for 16 hours with MRS (Difco) were used. After culturing using MRS, the bacterial cells were collected by centrifugation (8,000 g for 5 minutes) and then suspended into 0.8% sodium chloride solution. The resulting bacterial suspension (bacterial cell concentration of 1 to 2×10⁹ cfu/mL) was used as a starter. In the following Examples, S. thermophilus prepared by the same method as above was used as a starter, unless otherwise stated.

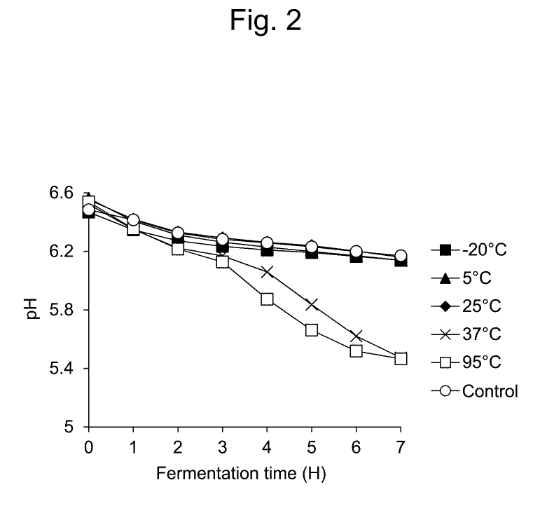

The pH of the fermentation liquid was measured over time. A decrease in the pH in the lactic acid bacteria culture medium indicates an increase in the amount of lactic acid production associated with the lactic acid bacterial fermentation and is used as an indicator of the progress degree of the lactic acid bacterial fermentation. The measurement results are shown in FIG. 2. When any of the lactose-NaOH solutions kept at −20° C., 5° C. and 25° C. was added, the pH decrease was not observed as compared to the control and the fermentation promoting effect was not observed. On the other hand, when the lactose-NaOH solution kept at 37° C. was added, the pH was greatly decreased as compared with the control, and the promotion of S. thermophilus fermentation was thus observed. The lactose-NaOH solution heated at 95° C. for 30 minutes exhibited a higher fermentation promoting effect than the lactose-NaOH solution kept at 37° C.

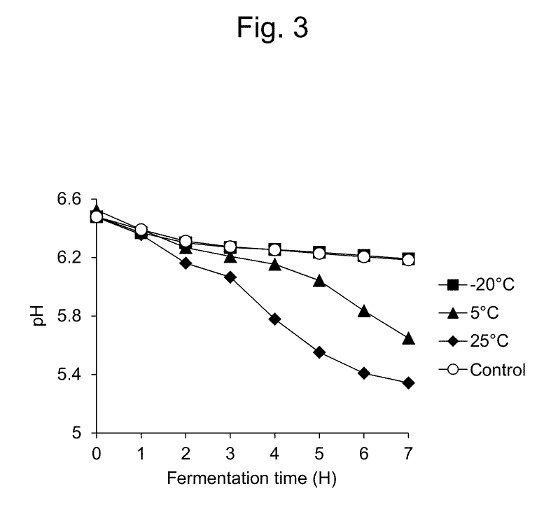

In addition, each of the lactose-NaOH solution kept at −20° C., 5° C. and 25° C. was added to UHT sterilized milk at 0.0125 (vol/wt), which is 5 times as much as the above-mentioned addition rate, and the otherwise same test as above was carried out. As a result, the lactose-NaOH solution kept at −20° C. exhibited no fermentation promoting effect. However, the lactose-NaOH solutions kept at 5° C. and 25° C. exhibited the fermentation promoting effect, and the fermentation promoting effect obtained by the lactose-NaOH solution kept at 25° C. was higher than that obtained by the lactose-NaOH solution kept a 5° C. (FIG. 3).

The above results show that a solution prepared by dissolving a sugar (lactose) in an alkaline solution (NaOH solution) and keeping or heat-treating at a temperature in the range of 5° C. or more has an effect of promoting fermentation of S. thermophilus. It also shows that the effect can be further enhanced by increasing the solution treating temperature.

### [Example 2] Effect of Low Concentration Lactose Solution Prepared with Low Concentration NaOH Solution

Lactose was dissolved in a 0.1% (wt/wt) NaOH solution to prepare a 0.1% (wt/wt) lactose solution. A sample of this 0.1% lactose solution was subjected to chilled storage at 5° C. (an unheated lactose-NaOH solution), and no coloration was observed. On the other hand, when a sample of the 0.1% lactose solution prepared was heated at 95° C. for 30 minutes to prepare a heated lactose-NaOH solution, the obtained solution turned slightly brown. Each of these lactose-NaOH solutions was added to UHT sterilized milk at 1% or 10% (vol/wt), and warmed to 43° C. After warming, S. thermophilus OLS3059 strain was inoculated as a starter into the UHT sterilized milk in an amount of 1% (vol/wt) and fermentation was started at 43° C. As a control, sterilized water was added to UHT sterilized milk at 1% or 10% (vol/wt), followed by warming to 43° C. Then S. thermophilus OLS3059 was inoculated as a starter thereinto in an amount of 1% (vol/wt), and fermentation was started at 43° C.

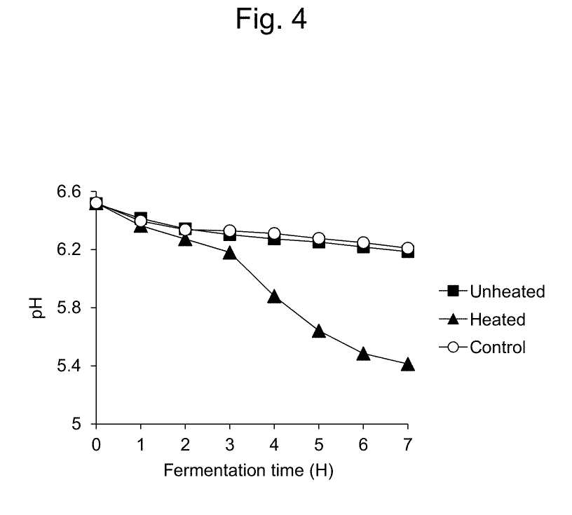

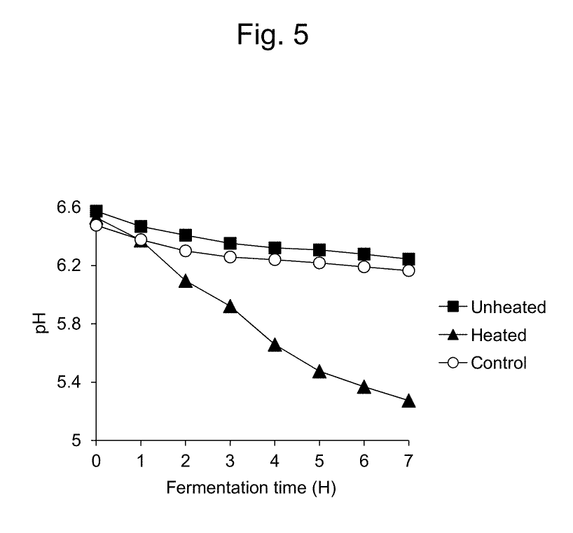

The pH of the fermentation liquid was measured over time. The measurement results are shown in FIG. 4 (addition rate: 1%) and FIG. 5 (addition rate: 10%). At both addition rates of the lactose-NaOH solution, the unheated lactose-NaOH solution exhibited no fermentation promoting effect, but the heated lactose-NaOH solution exhibited a fermentation promoting effect. This indicates that a sugar-alkaline solution prepared at a sugar concentration as low as 0.1% and an alkaline concentration as low as 0.1% can also exhibit a fermentation promoting effect when the solution was heated, and that the fermentation promoting effect can be obtained without problems at least at an addition rate of up to 10%.

### [Example 3] Correlation of Fermentation Promoting Effect with Sugar Concentration and NaOH Concentration

Lactose was dissolved in each of 0%, 0.8%, 1.6%, 8% and 27% (wt/wt) NaOH solutions to prepare a 25% (wt/wt) lactose solution. When a 27% NaOH solution was used, the lactose solution spontaneously generated heat and blackened after dissolution of lactose. The 25% lactose solution prepared was heat-treated at 95° C. for 30 minutes. The resulting heated lactose-NaOH solution was added to UHT sterilized milk at 0.01% (vol/wt), and warmed to 43° C. After warming, S. thermophilus OLS3059 strain was inoculated as a starter into the UHT sterilized milk in an amount of 1% (vol/wt) and the fermentation was started at 43° C. As a control, the same test was carried out by using UHT sterilized milk without the heated lactose-NaOH solution.

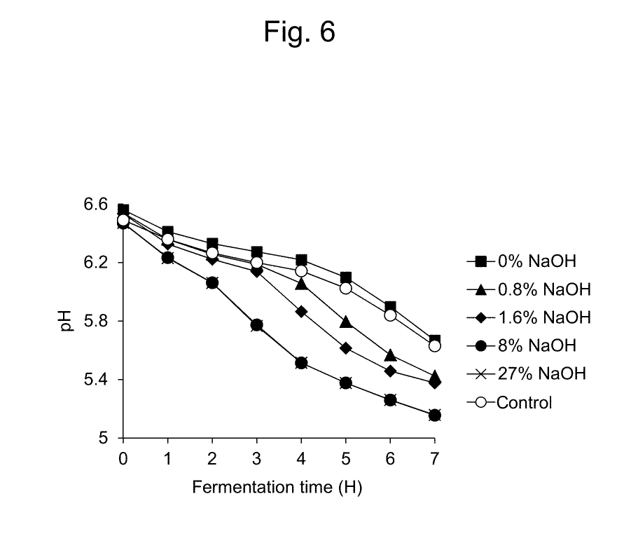

The pH of the fermentation liquid was measured over time. The measurement results are shown in FIG. 6. It was found that as the concentration of NaOH solution used for lactose dissolution was increased, the fermentation promoting effect increased. The lactose solution prepared by dissolving lactose in water (0% NaOH solution) exhibited no fermentation promoting effect even after heated. The lactose solutions prepared with 8% and 27% NaOH solutions exhibited almost the same levels of fermentation promoting effect.

Subsequently, three types of lactose-NaOH solutions different in the sugar concentration and the concentration of the alkaline solution were prepared and further tested. First, lactose was dissolved in a 27% (wt/wt) NaOH solution to prepare a 25% (wt/wt) lactose solution with a final NaOH concentration of 20.3% (hereinafter referred to as “25% Lac/27% NaOH”). In addition, lactose was dissolved in a 27% (wt/wt) NaOH solution to prepare a 50% (wt/wt) lactose solution with a final NaOH concentration of 13.5% (hereinafter referred to as “50% Lac/27% NaOH”). Further, lactose was dissolved in a 40% (wt/wt) NaOH solution to prepare a 70% (w/wt) lactose solution with a final NaOH concentration of 12% (hereinafter referred to as “70% Lac/40% NaOH”). All of these lactose-NaOH solutions spontaneously generated heat and blackened after dissolution.

Each of these lactose-NaOH solutions was heated at 95° C. for 30 minutes and added to UHT sterilized milk at 0.00325% (vol/wt), and warmed to 43° C. After warming, S. thermophilus OLS3059 strain was inoculated into the UHT sterilized milk in an amount of 1% (vol/wt) and fermentation was started at 43° C. As a control, the same test was carried out by using UHT sterilized milk without the heated lactose-NaOH solution.

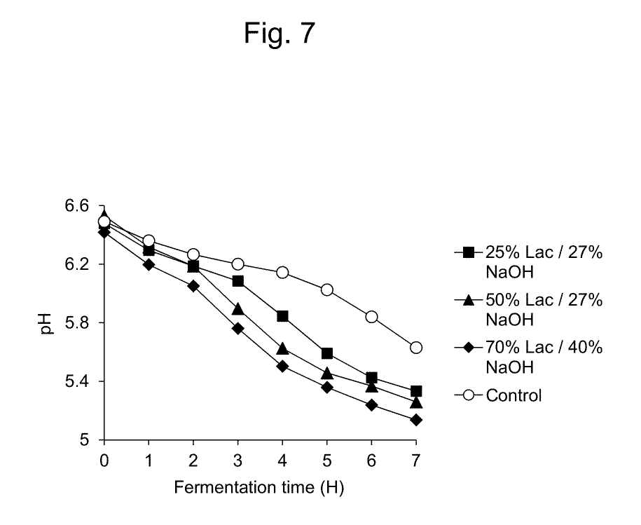

The pH of the fermentation liquid was measured over time. The measurement results are shown in FIG. 7. It was found that as the lactose concentration and the concentration of the NaOH solution used for lactose dissolution were increased, the fermentation promoting effect increased.

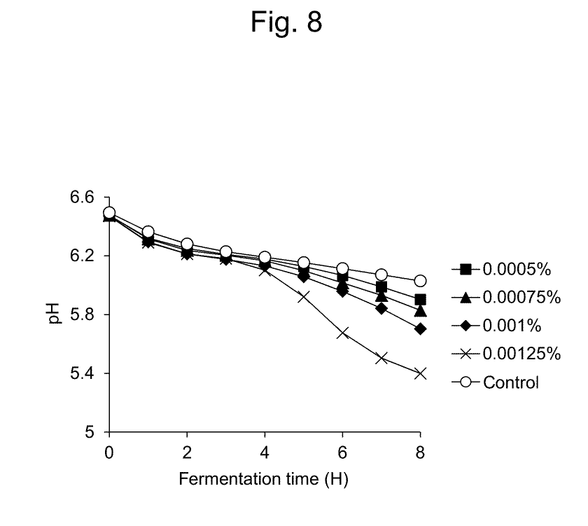

Then, “70% Lac/40% NaOH” heated at 95° C. for 30 minutes was added to UHT sterilized milk at 0.0005%, 0.00075%, 0.001% or 0.00125% (vol/wt), and warmed to 43° C. After warming, S. thermophilus OLS3059 strain was inoculated into the UHT sterilized milk in an amount of 1% (vol/wt) and fermentation was started at 43° C. As a control, the same test was carried out by using sterilized water instead of the heated lactose-NaOH solution. The pH of the fermentation liquid was measured over time. The measurement results are shown in FIG. 8. As a result, apparent fermentation promoting effect was obtained even at an addition rate of 0.0005% though the effect was at a low level. In addition, as the addition rate of 70% Lac/40% NaOH was increased, the fermentation promoting effect also increased. This revealed that a sugar-alkaline solution capable of promoting fermentation even when used at an extremely small addition rate can be produced by using increased concentrations of sugar and an alkaline solution and increased heating temperature.

### [Example 4] Influence of Types of Alkaline Solution on Fermentation Promoting Effect

A KOH solution, instead of the NaOH solution, was used as an alkaline solution. Specifically, lactose was dissolved in a 10% (wt/wt) KOH solution to prepare a 10% (wt/wt) lactose solution, and the resulting solution was heated at 95° C. for 30 minutes and then added to UHT sterilized milk at 0.025%, and warmed to 43° C. After warming, S. thermophilus OLS3059 strain was inoculated into the UHT sterilized milk in an amount of 1% (vol/wt) and fermentation was started at 43° C. As a control, the same test as above was carried out by using UHT sterilized milk without the heated lactose-KOH solution.

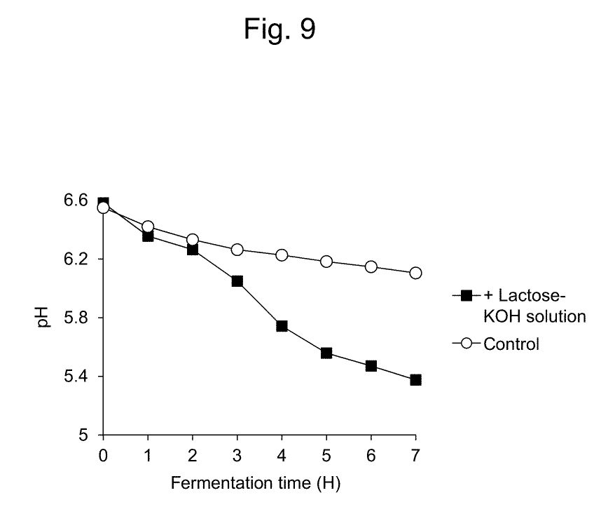

The pH of the fermentation liquid was measured (monitored) over time. The measurement results are shown in FIG. 9. It was revealed that a lactose solution prepared with a KOH solution (lactose-KOH solution) also promotes fermentation of S. thermophilus.

### [Example 5] Influence of Types of Sugar on Fermentation Promoting Effect

A similar test was carried out by using different types of sugar instead of lactose. As a monosaccharide, glucose, galactose, fructose, arabinose, rhamnose, xylose, xylitol, mannitol or sorbitol was used. As a disaccharide, lactulose, sucrose or trehalose was used. As an oligosaccharide, galactooligosaccharide or fructooligosaccharide was used, and as a polysaccharide, dextrin was used. In a 25% (wt/wt) NaOH solution, the sugar was dissolved at a concentration of 12.5% (wt/wt) to prepare a sugar-NaOH solution, and the resulting solution was heated at 95° C. for 30 minutes. The observation of the appearance of the sugar-NaOH solution after heated was carried out. After heating, each of the sugar solutions was individually added to UHT sterilized milk at 0.035% (vol/wt). The UHT sterilized milk was heated to 43° C. and then S. thermophilus 1131 strain was inoculated thereinto in an amount of 1% (vol/wt), and fermentation was started at 43° C. As a control, the same test as above was carried out by using sterilized water instead of the heated sugar-NaOH solution.

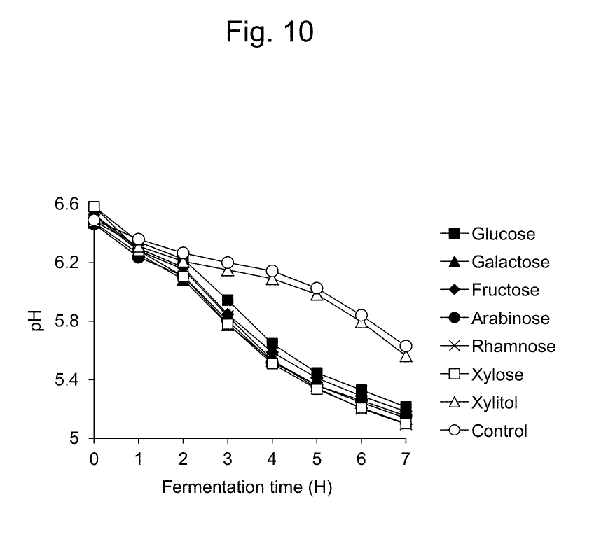

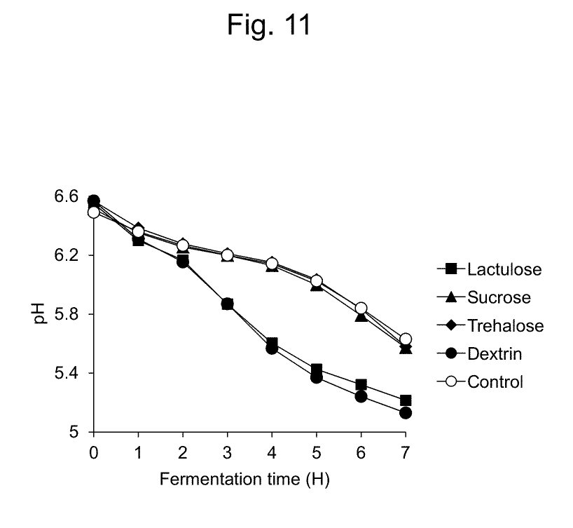

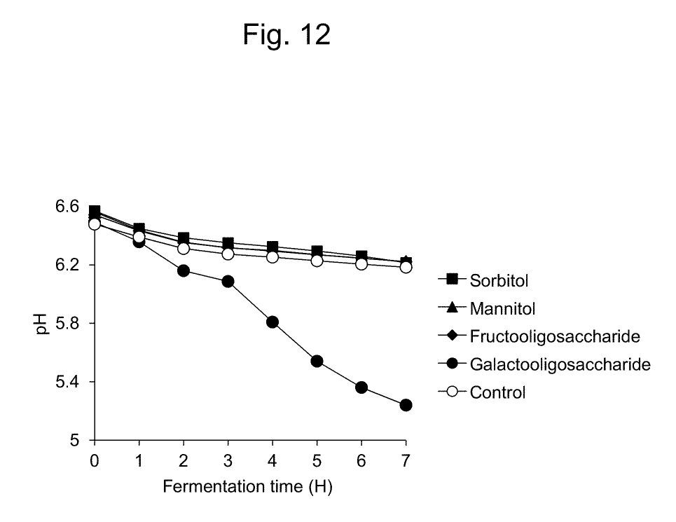

The pH of the fermentation liquid was measured (monitored) over time. The measurement results are shown in FIGS. 10 to 14. When glucose, galactose, fructose, arabinose, rhamnose or xylose as a monosaccharide, lactulose as a disaccharide, or galactooligosaccharide or dextrin as a polysaccharide was used, a fermentation promoting effect was obtained (FIGS. 10, 11 and 12).

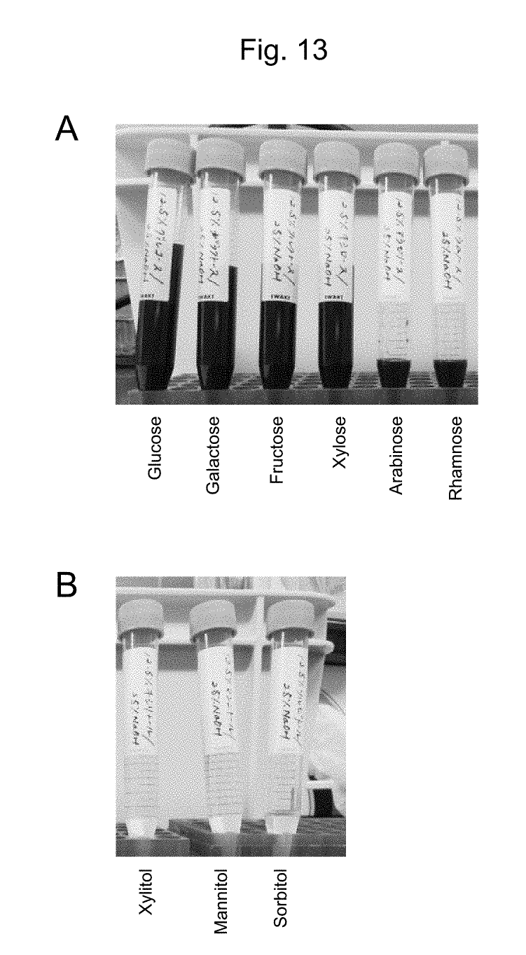

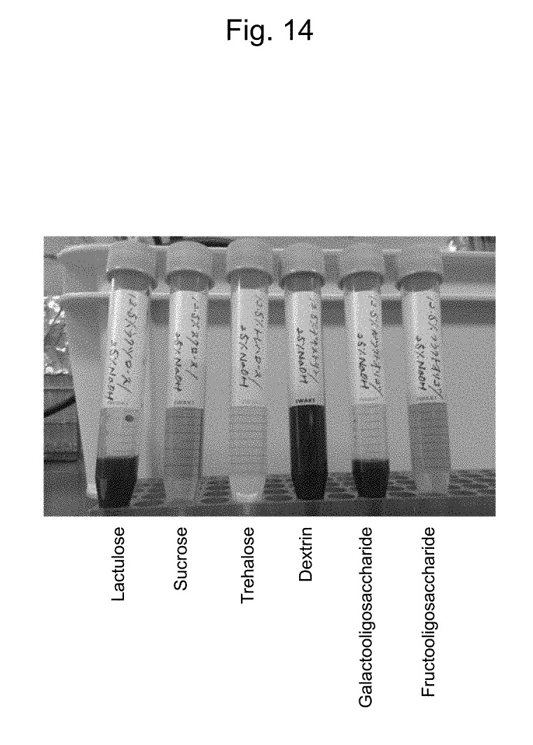

The color tone of each of sugar-NaOH solutions after heated is shown in FIGS. 13 and 14. The monosaccharides (reducing sugars) exhibiting the fermentation promoting effect blackened by heating after dissolution in NaOH solution, but the monosaccharides (non-reducing sugars) exhibiting no fermentation promoting effect only turned slightly brown or remained colorless and transparent (FIG. 13). Similar results were obtained with the disaccharides and the polysaccharides (FIG. 14).

### [Example 6] Fermentation Promoting Effect of Alkaline Solution Comprising Saccharide-Containing Food Material

The fermentation promoting effect of sugar-alkaline solution was similarly tested using a fruit juice instead of sugars. Fruit juice is known to contain a high level of sugars such as fructose. The fruit juices used were 100% grape juice (Seven & i Holdings Co., Ltd.; Carbohydrate content of 24.7 g/200 ml), 100% grapefruit juice (Dole Food Company, Inc.; Carbohydrate content of 16.8 g/200 ml), 100% orange juice (Seven & i Holdings Co., Ltd.; Carbohydrate content of 20.7 g/200 ml), and 100% apple juice (Seven & i Holdings Co., Ltd.; Carbohydrate content of 22.1 g/200 ml). In a control group, the fruit juice was heated at 95° C. for 15 minutes. In the test group, NaOH was added to the fruit juice at 10% (wt/wt) and then the resultant was heated at 95° C. for 15 minutes.

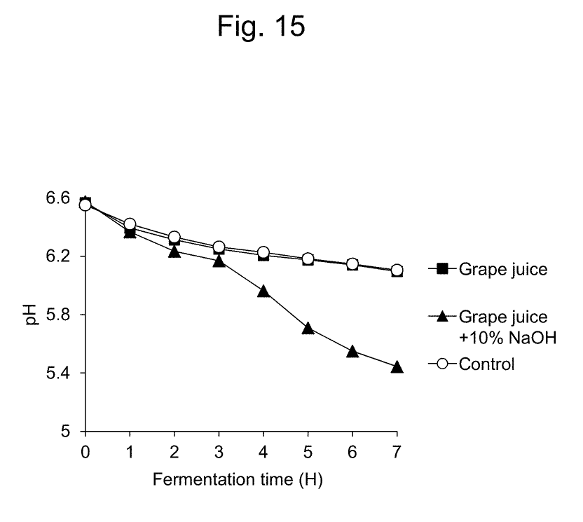

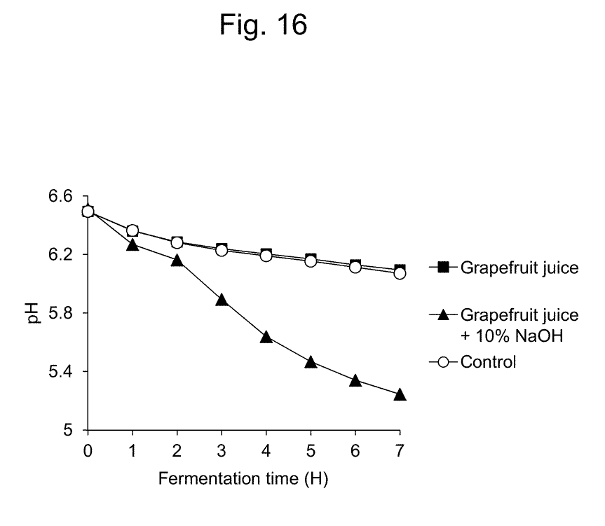

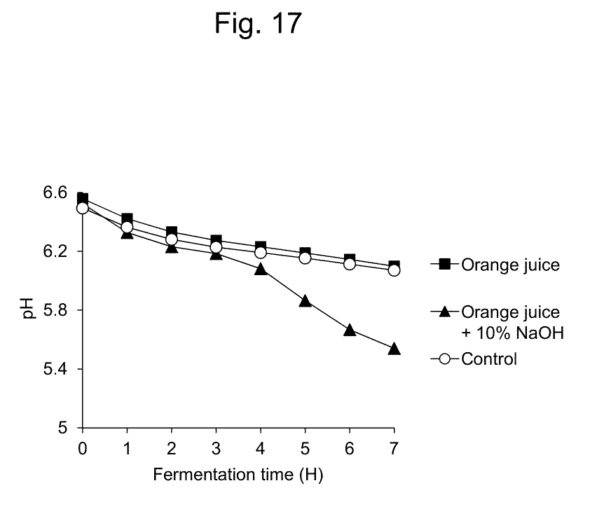

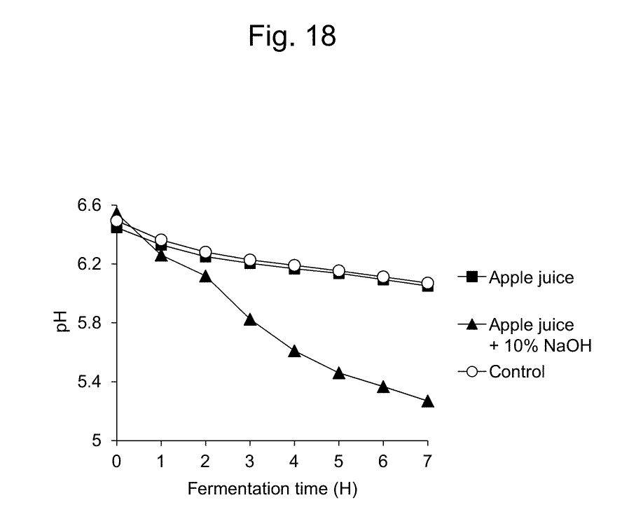

Each fruit juice after heated was added to UHT sterilized milk at 0.005% (vol/wt), and warmed to 43° C. After warming, S. thermophilus OLS3059 strain was inoculated into the UHT sterilized milk in an amount of 1% (vol/wt) and fermentation was started at 43° C. In addition, as a control, the same test as above was carried out by using sterilized water instead of the fruit juices heated after adding NaOH thereto. The pH of the fermentation liquid was measured over time. The measurement results are shown in FIGS. 15 to 18. For all the fruit juices tested, the fruit juices heated without NaOH added exhibited no fermentation promoting effect, but the fruit juices heated after adding NaOH thereto exhibited a fermentation promoting effect (FIGS. 15 to 18).

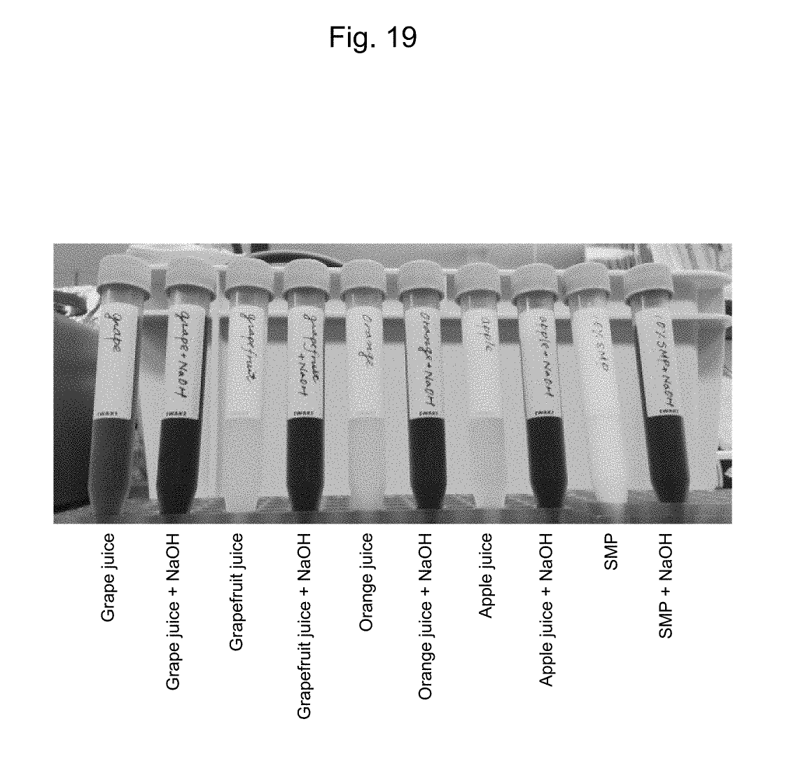

All the fruit juices blackened by heating after adding NaOH thereto (FIG. 19). Although the grape juice was originally blackish, it noticeably blackened by the heat-treatment after adding NaOH.

Next, the fermentation promoting effect of sugar-alkaline solution was similarly tested using a reconstituted skim milk (SMP) instead of sugar. The reconstituted skim milk is known to be prepared by dissolving a powdered skim milk (dry powder) in water or the like and to contain lactose.

NaOH was added to a reconstituted skim milk (from a powdered skim milk manufactured by Meiji Co., Ltd.) at a concentration of 5% (wt/wt) to prepare a 10% reconstituted skim milk and the reconstituted skim milk was heat-treated at 95° C. for 15 minutes. The reconstituted skim milk heated after adding NaOH thereto was added to UHT sterilized milk at 0.005% (vol/wt), and warmed to 43° C. After warming, S. thermophilus OLS3059 strain was inoculated into the UHT sterilized milk in an amount of 1% (vol/wt) and fermentation was started at 43° C. For comparison, the same test as above was carried out by using a reconstituted skim milk heated without NaOH instead of the reconstituted skim milk heated after adding NaOH thereto. In addition, as a control, the same test as above was carried out by using sterilized water instead of the reconstituted skim milk heated after adding NaOH thereto.

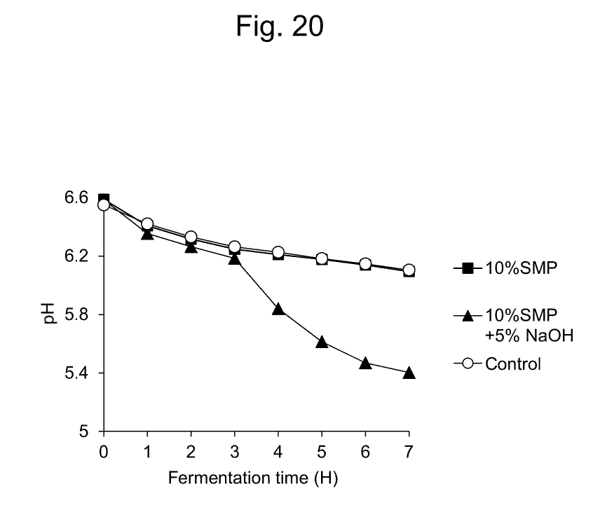

The pH of the fermentation liquid was measured over time. The measurement results are shown in FIG. 20. The reconstituted skim milk heated without NaOH exhibited no fermentation promoting effect, but the reconstituted skim milk heated after adding NaOH thereto exhibited a fermentation promoting effect. The reconstituted skim milk having NaOH added thereto blackened without precipitation or the like by heating (FIG. 19).

The above results revealed that compositions prepared by dissolving a food material containing a sugar such as fructose and lactose, for example, a fruit juice or a reconstituted skim milk, in an alkaline solution and heating it can also promote the S. thermophilus fermentation.

### [Example 7] Fermentation Promoting Effect of Sugar-Alkaline Solution on Various Strains of Streptococcus thermophilus

As described in Example 3, “50% Lac/25% NaOH” was prepared and heated at 95° C. for 30 minutes. The resulting solution was added to UHT sterilized milk at 0.005% (vol/wt), and warmed to 43° C. After warming, S. thermophilus was inoculated into the UHT sterilized milk and fermentation was started at 43° C. As a control, the same test as above was carried out by using UHT sterilized milk without the “50% Lac/25% NaOH”.

As S. thermophilus, six strains of S. thermophilus OLS3059 strain (accession number FERM BP-10740), S. thermophilus OLS3294 strain (accession number NITE P-77), S. thermophilus OLS3289 strain (ATCC 19258), S. thermophilus OLS3469 strain (IFO 13957/NBRC 13957), S. thermophilus OLS3058 strain and S. thermophilus OLS3290 strain (accession number FERM BP-19638) were used individually.

The preparation of six strains of S. thermophilus was carried out in the same manner as that for S. thermophilus OLS3059 strain described in Example 1. The strains OLS3059 and OLS3294 were inoculated into UHT sterilized milk in an amount of 1% (vol/wt), the strains OLS3289, OLS3469 and OLS3058 were inoculated into UHT sterilized milk in an amount of 1.5% (vol/wt) and the strain OLS3290 was inoculated into UHT sterilized milk in an amount of 3% (vol/wt), so that an equal number of cells were added to each milk.

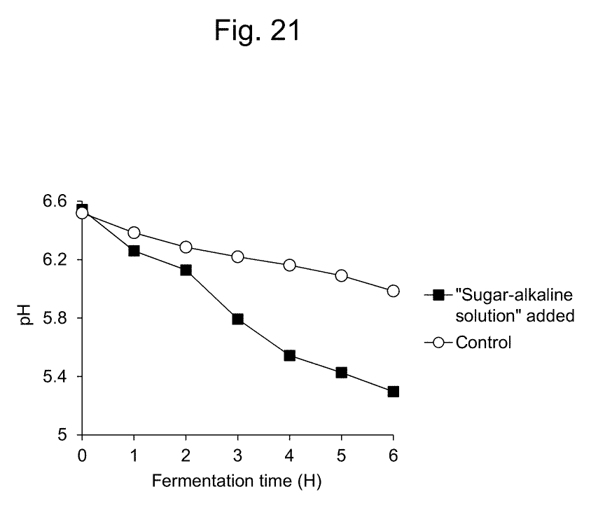

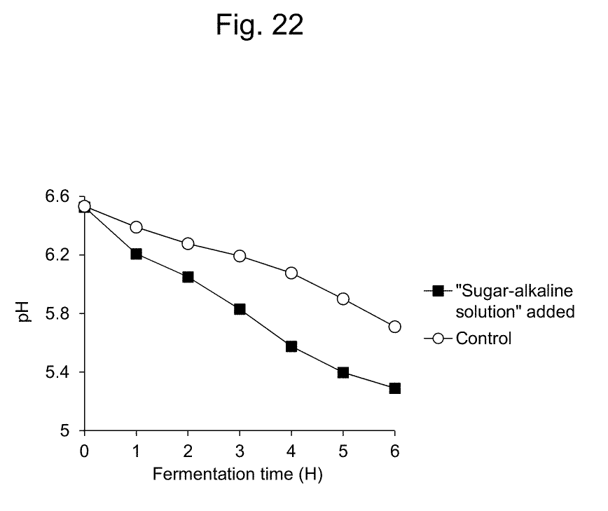

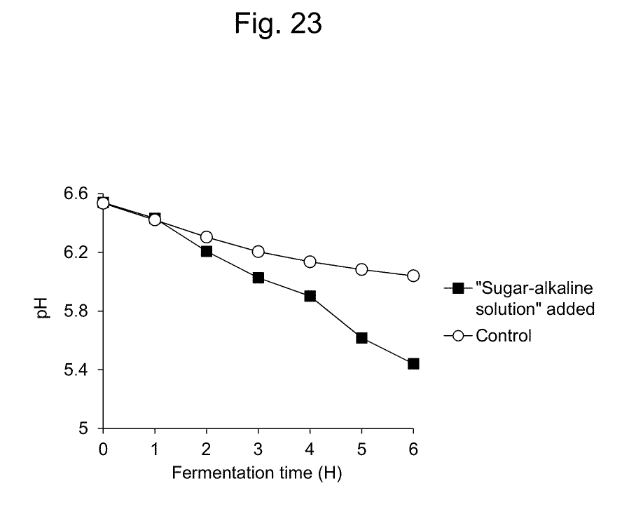

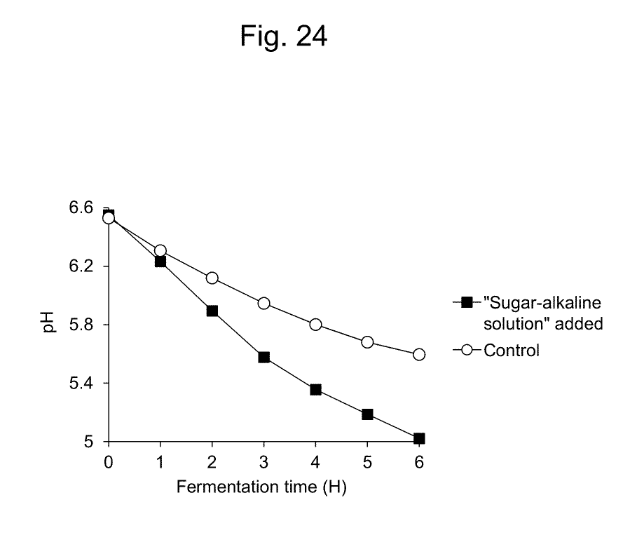

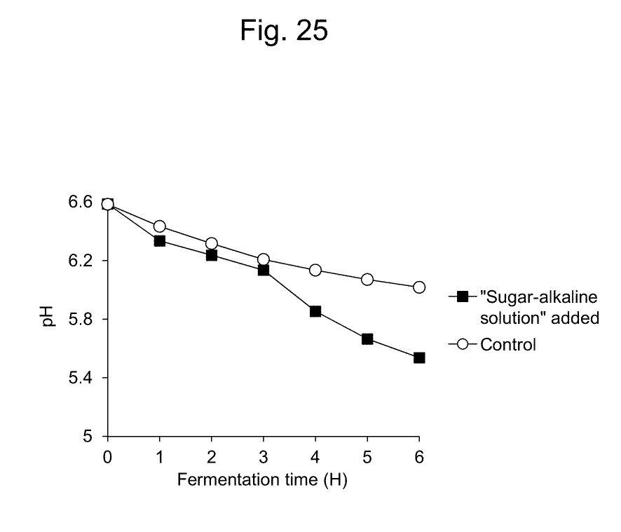

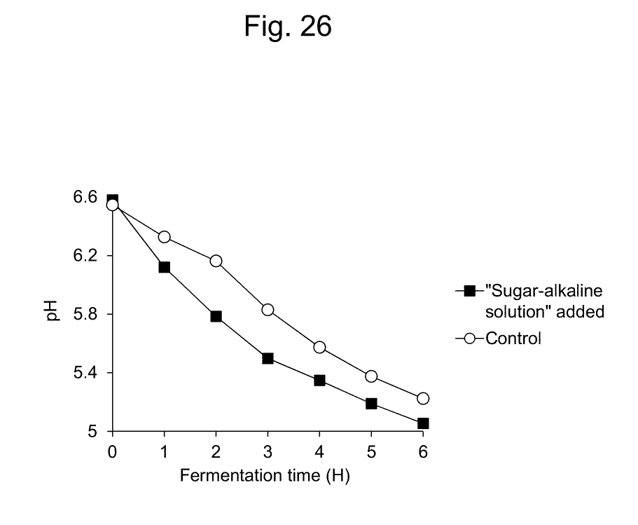

The pH of the fermentation liquid was measured (monitored) over time. The measurement results are shown in FIGS. 21 to 26. All strains of S. thermophilus tested were found to exhibit a fermentation promoting effect. This indicates that the sugar-alkaline solution according to the present invention exerts a fermentation promoting effect on various strains of S. thermophilus.

### [Example 8] Fermentation Promoting Effect of Sugar-Alkaline Solution in Mixed Fermentation of S. thermophilus and L. bulgaricus

In the present Example, the fermentation promoting effect of sugar-alkaline solution on Streptococcus thermophilus (S. thermophilus) was tested in a mixed culture (co-culture) using S. thermophilus and Lactobacillus bulgaricus (or Lactobacillus delbrueckii subsp. bulgaricus; L. bulgaricus), which are used in producing yogurt.

As described in Example 3, “50% Lac/25% NaOH” was prepared and heated at 95° C. for 30 minutes. The resulting heated sugar-alkaline solution was added to UHT sterilized milk at 0.005% (vol/wt), and warmed to 43° C. After warming, the UHT sterilized milk was inoculated with 1% (vol/wt) of S. thermophilus OLS3059 strain and 0.2% (vol/wt) of L. bulgaricus OLL1073 R-1 strain (accession number FERM BP-10741) and fermentation was started at 43° C. As a control, fermentation was also carried out by using UHT sterilized milk without the heated sugar-alkaline solution. The preparation of L. bulgaricus OLL1073R-1 strain was carried out in the same manner as that for S. thermophilus OLS3059 strain described in Example 1.

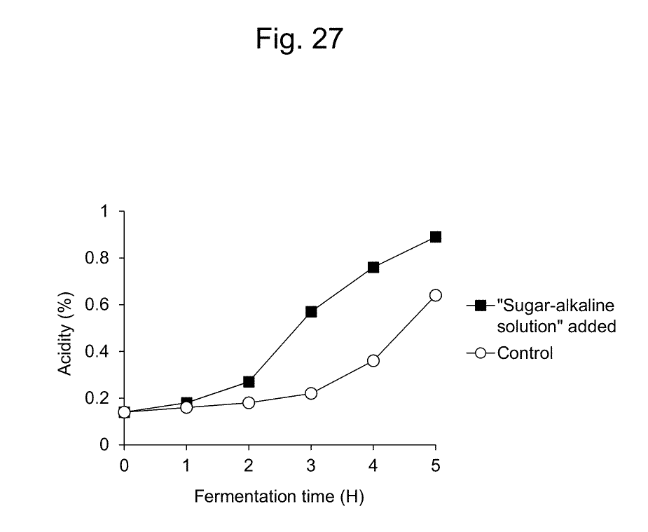

The acidity of the fermentation liquid was measured over time. Specifically, 0.5 mL of phenolphthalein was added to 9 g of the fermentation liquid and then 0.1 N NaOH was added thereto until the fermentation liquid turned pale red, for neutralization titration. The lactic acid concentration (%) of the fermentation liquid was calculated assuming that the whole amount of 0.1 N NaOH required for the titration corresponds to the amount of lactic acid, and used as the acidity. The results are shown in FIG. 27. The addition of the heated sugar-alkaline solution largely increased the acidity of the fermentation liquid as compared with the control, indicating that the fermentation had been promoted.

Further, the L-lactic acid concentration of the fermentation liquid was measured by high performance liquid chromatography (HPLC). The conditions used for HPLC measurement are shown in Table 1.

TABLE 1
HPLC measurement conditions

| | |
|---|---|
| HPLC | Shimadzu SCL-10A SP system (Shimadzu) |
| Column | Sumichiral OA 5000 column, 4.6 mm × 150 |
| | mm, 5 μm (Sumika Chemical Analysis Service) |
| Detector | UV (254 nm) |
| Mobile phase | 2 mM CuSO₄ (II)•5H₂O and 5% 2-propanol |
| Temperature | 40° C. |
| Flow rate of mobile phase | 1.0 mL/min |
| Injection volume | 10 μL |

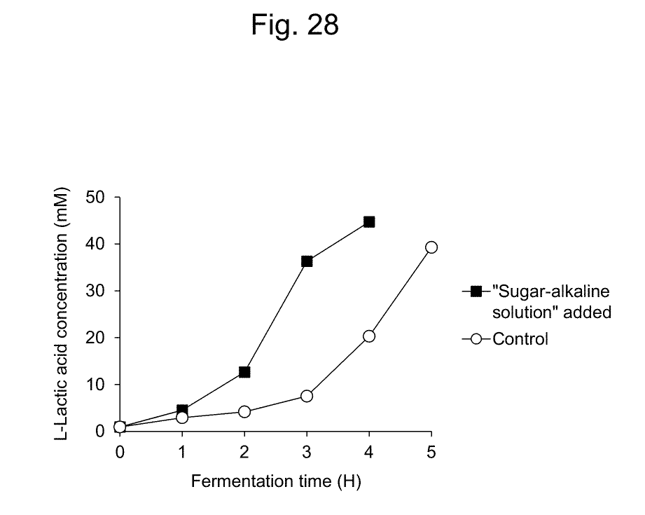

It was found that the addition of the heated sugar-alkaline solution promoted the production of L-lactic acid (FIG. 28). S. thermophilus is known to produce L-lactic acid and L. bulgaricus is known to produce D-lactic acid (Microorganisms, Vol. 6, No. 1, p. 2-3 (1990); and Modern Media, Vol. 57, No. 10, p. 277-287 (2011)). Therefore, the results indicating that the production of L-lactic acid was promoted show that the fermentation by S. thermophilus was promoted also in mixed fermentation (mixed culture) of S. thermophilus and L. bulgaricus.

This indicates that the sugar-alkaline solution of the present invention can be also used for promoting fermentation in the production of yogurt.

### [Example 9] Influence of Sugar-Alkaline Solution on Taste in Yogurt Fermentation

As described in Example 3, “50% Lac/25% NaOH” was prepared and heated at 95° C. for 30 minutes. This heated sugar-alkaline solution was used to prepare yogurt in accordance with the mixing proportions indicated in Table 2. First, the ingredients other than the yeast extract and the heated sugar-alkaline solution (Table 2) were mixed to prepare a yogurt base and the yogurt base was sterilized at 95° C., and cooled to 40 to 45° C. Thereafter, the heated sugar-alkaline solution was added thereto (sugar-alkaline solution group). For comparison of taste, the test group in which the yeast extract instead of the heated sugar-alkaline solution was added as a fermentation promoting agent (yeast extract group) and the control group in which neither the heated sugar-alkaline solution nor the yeast extract was added were also prepared (Table 2). They were sterilized at 90° C. prior to inoculation with a starter.

TABLE 2
Mixing Proportion of ingredients in yogurt

| | Mixing Proportions (%) | | |
|---|---|---|---|
| | Control | Yeast extract | Sugar-alkaline |
| Ingredients | group | group | solution group |
| UHT sterilized milk | 77.0 | 77.0 | 77.0 |
| Powdered skim milk | 2.8 | 2.8 | 2.8 |
| Whey protein concentrate | 0.2 | 0.2 | 0.2 |
| (WPI) | | | |
| Sucrose | 4.5 | 4.5 | 4.5 |
| Stevia | 0.0075 | 0.0075 | 0.0075 |
| Yeast extract | — | 0.01 | — |
| Sugar-alkaline solution | — | — | 0.0025 |
| Starter | 0.15 | 0.05 | 0.05 |
| Water | 15.34 | 15.43 | 15.44 |

A starter was prepared by mixing L. bulgaricus OLL1255 strain (accession number NITE BP-76) (bacterial cell concentration of 1×10⁹ cfu/mL) and S. thermophilus OLS3294 strain (accession number NITE P-77) (bacterial cell concentration of 3×10⁹ cfu/mL), culturing them in high cell concentrations and cryopreserving them. This starter was inoculated in an amount of 0.05% (vol/wt) for the yeast extract group and the sugar-alkaline solution group and in an amount of 0.15% (vol/wt) for the control group. After inoculating the starter, fermentation was carried out at 43° C. until the acidity reached 0.75%, followed by cooling at 5° C., to prepare yogurt.

The prepared yogurt was evaluated for taste by five expert panels trained in sensory evaluation of yogurt. Each of the evaluation items: curd physical property, acidity, sweetness, and unpleasant taste of the yogurt was scored on a 5-point scale. The average score in the control group of each evaluation item was set as 1, and the relative value of the average score in each of the yeast extract group and the sugar-alkaline solution group was calculated relative to that of the control group. The curd physical property was evaluated in view of “smoothness” and “firmness”. The results are shown in Table 3.

TABLE 3

| | Control | Yeast extract | Sugar-alkaline |
|---|---|---|---|
| | group | group | solution group |
| Physical property of curd | 1.0 | 1.0 | 1.1 |
| Sourness | 1.0 | 1.2 | 1.2 |
| Sweetness | 1.0 | 0.9 | 0.9 |
| Unpleasant taste such as | 1.0 | 1.8 | 1.0 |
| bitterness and harsh taste | | | |

As shown in Table 3, there was little difference in the physical property, sourness and sweetness among the yogurts of these groups. The yogurt of the yeast extract group was markedly inferior in terms of the unpleasant taste, whereas the yogurt of the sugar-alkaline solution group had no difference in the unpleasant taste from that of the control group. As seen in the details of the evaluation results on the unpleasant taste, three panels clearly sensed the unpleasant taste in the yogurt prepared adding the yeast extract, whereas no panel sensed the unpleasant taste in the yogurt prepared adding the heated sugar-alkaline solution as well as the yogurt prepared adding neither the yeast extract nor the heated sugar-alkaline solution (Control). Thus, the heated sugar-alkaline solution was superior to the yeast extract in that it has little adverse influence on the taste of yogurt.

In addition, in the fermentation of the yogurt in which the heated sugar-alkaline solution or the yeast extract was added, the time for completion of the fermentation was shortened by more than 2 hours compared with the control, even though the amount of the starter inoculated was only ⅓ of that in the control. This indicates that the heated sugar-alkaline solution also notably promotes the fermentation for yogurt production.

### INDUSTRIAL APPLICABILITY

According to the present invention, materials capable of promoting the fermentation by S. thermophilus even when used in a very small amount, can be provided. The materials can be used to shorten the fermentation process in the production of fermented foods, with little influence (due to unpleasant taste or the like) on the taste of fermented foods such as fermented milk.

All publications, patents and patent applications cited in the present specification are incorporated herein by reference in their entirety.

1. A fermentation promoting agent for Streptococcus thermophilus,
comprising a solution prepared by exposing an alkaline solution comprising a reducing sugar to a temperature in the range of greater than or equal to 5° C. and less than or equal to 135° C. to induce a color reaction of sugar.

2. The fermentation promoting agent according to claim 1, wherein said temperature is 35° C. or higher.

3. The fermentation promoting agent according to claim 1, wherein said solution is prepared by heating the alkaline solution comprising a reducing sugar at to a temperature ranging from 35 to 100° C.

4. The fermentation promoting agent according to claim 1, wherein the alkaline solution comprising a reducing sugar comprises from 0.05 to 80 wt % of the reducing sugar.

5. The fermentation promoting agent according to claim 1, wherein the reducing sugar is selected from the group consisting of glucose, galactose, fructose, arabinose, rhamnose, xylose, lactose, lactulose, galactooligosaccharide and dextrin.

6. The fermentation promoting agent according to claim 1, wherein the alkaline solution comprising a reducing sugar comprises from 0.05 to 30 wt % of a hydroxide.

7. The fermentation promoting agent according to claim 1, wherein the alkaline solution comprising a reducing sugar comprises at least one of sodium hydroxide and potassium hydroxide.

8. The fermentation promoting agent according to claim 7, wherein the reducing sugar is lactose.

9. The fermentation promoting agent according to claim 1, wherein the alkaline solution comprising a reducing sugar comprises a food material comprising a reducing sugar.

10. The fermentation promoting agent according to claim 9, wherein said food material is at least one of a fruit juice and a reconstituted skim milk.

11. A method for producing a fermentation promoting agent for Streptococcus thermophilus, comprising exposing an alkaline solution comprising a reducing sugar to a temperature in the range of greater than or equal to 5° C. and less than or equal to 135° C. to induce a color reaction of sugar, thereby preparing a solution having a fermentation promoting effect on Streptococcus thermophilus.

12. The method according to claim 11, wherein said temperature is 35° C. or higher.

13. The method according to claim 11, wherein said solution is prepared by heating the alkaline solution comprising a reducing sugar at to a temperature ranging from 35 to 100° C.

14. The method according to claim 11, wherein the alkaline solution comprising a reducing sugar comprises from 0.05 to 80 wt % of the reducing sugar.

15. The method according to claim 11, wherein the reducing sugar is selected from the group consisting of glucose, galactose, fructose, arabinose, rhamnose, xylose, lactose, lactulose, galactooligosaccharide and dextrin.

16. The method according to claim 11, wherein the alkaline solution comprising a reducing sugar comprises from 0.05 to 30 wt % of a hydroxide.

17. The method according to claim 11, wherein the alkaline solution comprising a reducing sugar comprises at least one of sodium hydroxide and potassium hydroxide.

18. The method according to claim 17, wherein the reducing sugar is lactose.

19. The method according to claim 11, wherein the alkaline solution comprising a reducing sugar comprises a food material comprising a reducing sugar.

20. The method according to claim 19, wherein said food material is at least one of a fruit juice and a reconstituted skim milk.

21. A method for promoting Streptococcus thermophilus fermentation, comprising adding the fermentation promoting agent according to claim 1 to a fermentation substrate and culturing Streptococcus thermophilus in/on the fermentation substrate to ferment the fermentation substrate.

22. The method according to claim 21, wherein Streptococcus thermophilus and Lactobacillus bulgaricus are mixed-cultured in/on the fermentation substrate.

23. A method for producing a milk-fermented food, comprising fermenting a fermentation substrate comprising milk or a milk-derived product with the method according to claim 22.

24. The method according to claim 23, wherein the milk-fermented food is fermented milk.
